(19) United States
(12) Patent Application Publication
(10) Pub. No.: US 2022/0267726 A1
(43) Pub. Date: Aug. 25, 2022

(54) ORDERED PROCESSING OF BLOOD PRODUCTS TO PRODUCE THERAPEUTICALLY ACTIVE CELLS

(71) Applicant: GPB SCIENTIFIC, INC., Richmond, VA (US)

(72) Inventors: Anthony WARD, Rancho Santa Fe, CA (US); Roberto CAMPOS-GONZALEZ, Carlsbad, CA (US); Alison SKELLEY, Riverside, CA (US); Khushroo GANDHI, Palo Alto, CA (US); Yasna BEHMARDI, Mission Viejo, CA (US)

(21) Appl. No.: 17/627,353

(22) PCT Filed: Jul. 17, 2020
(86) PCT No.: PCT/US2020/042634

§ 371 Date: Jan. 14, 2022

Related U.S. Application Data
(60) Provisional application No. 62/875,942, filed on Jul. 18, 2019.

(51) Int. Cl. C12N 5/0783; A61K 35/17

(52) U.S. Cl. CPC C12N 5/0636; A61K 35/17; C12N 2501/2307; C12N 2501/2315; C12N 2501/515; C12N 2502/1114; C12N 2502/1128; C12N 2502/115

(57) ABSTRACT

Provided are methods for processing a blood related sample comprising: (a) providing a blood related sample comprising one or more target cells, platelet cells, red blood cells; and (b) reducing a number of the platelet cells in the blood related sample while maintaining a ratio of the red blood cells to the one or more target cells above a critical threshold to produce a reduced platelet blood related sample comprising the one or more target cells. Also described herein are cell compositions produced by applying the methods described herein.

### CROSS-REFERENCE

This application claims the benefit of U.S. Provisional Application No. 62/875,942, filed Jul. 18, 2019, which application is incorporated herein by reference.

### BACKGROUND

Breakthroughs in cell-based therapies have driven an ever-expanding number of clinical trials and US Food and Drug administration approvals of therapeutic cells for the treatment of disease. In the translation of pre-clinical discoveries into cell-based therapies, the effective and timely manufacture of therapeutic cells remains a barrier to the successful implementation of cell therapies. The first hurdle in this process is the collection of target cells to be used to generate a therapeutic cell product (e.g. T cells for chimeric antigen receptor T cell therapy). Generally, attention to target cell collection focuses on obtaining a threshold number of desired target cells to serve as input for cellular engineering efforts that yield the therapeutic cell product. However, even if target cells are successfully collected above a desired threshold, an effective therapeutic cell product may yet fail to be obtained due to the nature of the isolated target cell composition or collection process that results in attenuating the efficacy of downstream processing and or the therapeutic cell product itself.

### SUMMARY

Provided herein are compositions, methods, and systems utilizing an ordered processing of a blood sample to isolate target cells. The ordered processing provides for the generation of unique target cell compositions that enable the use of the target cells for the effective engineering of therapeutic cell products. Generally, the disclosed compositions, methods, and systems maintain or utilize erythrocytes (i.e. red blood cell) in a sample while sequentially eliminating non-target cells, cell fragments (e.g. platelets), and other factors to provide a means of obtaining target cells (e.g., leukocytes) that are especially well suited for cell engineering and therapeutic use (e.g. chimeric antigen receptor T cell therapy, adoptive immune cell therapies). Accordingly, compositions, methods, and systems disclosed here are useful for the generation and manufacture of cell-based therapies.

Provided are methods and compositions for use in processing a blood related sample comprising: (a) providing a blood related sample comprising one or more target cells, platelet cells, red blood cells; and (b) reducing a number of the platelet cells in the blood related sample while maintaining a ratio of the red blood cells to the one or more target cells greater than about 50:1 to produce a reduced platelet blood related sample comprising the one or more target cells.

In some embodiments, the blood related sample comprises a hematocrit of greater than about 2%. In some embodiments, the blood related sample comprises a hematocrit of greater than about 4%. In some embodiments, the blood related sample comprises a hematocrit of less than about 30%.

In some embodiments, the blood related sample is a leukapheresis product. In some embodiments, the reduced platelet blood related sample comprises a ratio of platelets to target cells of less than about 500:1. In certain embodiments, the reduced platelet blood related sample comprises a ratio of platelets to target cells of less than about 100:1.

In certain embodiments, the reduced platelet blood related sample comprises a ratio of platelets to target cells of less than about 10:1.

In some embodiments, the reduced platelet blood related sample comprises a ratio of platelets to target cells of less than about 5:1. In some embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1.

In some embodiments, the method further comprises removing one or more non-target cells from the blood related sample and/or the reduced platelet blood related sample. In certain embodiments, the one or more non-target cells comprise immune suppressive cells. In certain embodiments, the immune suppressive cells are regulatory T cells. In certain embodiments, the immune suppressive cells are regulatory B cells. In certain embodiments, the immune suppressive cells comprise myeloid derived suppressor cells.

In some embodiments, the non-target cells are removed by an affinity-based method. In certain embodiments, the affinity-based method targets a molecule on the cell surface of the non-target cells. In some embodiments, the affinity-based method comprises the use of an antibody. In certain embodiments, the antibody is conjugated to biotin, streptavidin, a fluorescent moiety, or a magnetic material.

In some embodiments, the methods comprise adding an anticoagulant to the blood related sample. In some embodiments, the blood related sample is a human blood related sample. In some embodiments, the blood related sample is collected from an individual afflicted with a cancer or a tumor or an HLA matched individual to the individual afflicted with a cancer or a tumor. In certain embodiments, the blood related sample is collected from an individual afflicted with a cancer or a tumor. In some embodiments, the reducing the number of the platelet cells from the blood related sample comprises use of a method which uses an affinity reagent, a deterministic lateral displacement method, a method which uses a density media, an acoustophoretic method, or a dielectrophoretic method. In certain embodiments, the reducing the number of the platelet cells from the blood related sample uses a method comprising deterministic lateral flow.

In some embodiments, the method further comprises isolating the one or more target cells from the reduced platelet blood related sample to produce one or more isolated target cells. In some embodiments, the one or more target cells comprise peripheral blood mononuclear cells. In some embodiments, the one or more target cells comprise a stem cell, a lymphoid cell, or a myeloid cell. In certain embodiments, the stem cell is a hematopoietic stem cell. In certain embodiments, the lymphoid cell is a T cell. In certain embodiments, the T cell displays a naïve phenotype. In certain embodiments, the T cell displays a central memory phenotype. In certain embodiments, the lymphoid cell is a natural killer cell or a natural killer T cell. In certain embodiments, the myeloid cell is a dendritic cell. In certain embodiments, the myeloid cell is a macrophage cell. In some embodiments, the one or more target cells are isolated by a method which uses an affinity reagent, a deterministic lateral displacement method, a method which uses a density media, an acoustophoretic method, or a dielectrophoretic method. In some embodiments, the one or more target cells are isolated by a method which uses an affinity reagent. In some embodiments, the one or more target cells are isolated using deterministic lateral displacement.

In some embodiments, the method further comprises culturing the one or more target cells of the reduced platelet blood related sample or the one or more isolated target cells. In some embodiments, the method further comprises genetically engineering the one or more target cells of the reduced platelet blood related sample or the one or more isolated target cells. In certain embodiments, the genetic engineering comprises rendering the one or more target cells transgenic for a chimeric antigen receptor. In certain embodiments, the genetic engineering comprises rendering the one or more target cells transgenic for a recombinant T cell receptor. In some embodiments, the method further comprises comprising activating the one or more target cells prior to or after the genetic engineering.

Further provided are compositions, for example, provided are cell populations comprising one or more target cells, platelet cells and red blood cells, the target cells at a ratio of platelets to target cells less than about 500:1 and at a ratio of red blood cells to target cells of greater than about 50:1. In some embodiments, the target cells comprise human cells. In some embodiments, the target cells, platelet cells, and red blood cells comprise human cells.

In some embodiments, the ratio of platelets to target cells is less than about 100:1. In some embodiments, the ratio of platelets to target cells is less than about 10:1. In some embodiments, the ratio of platelets to target cells is less than about 5:1. In some embodiments, the ratio of red blood cells to target cells is greater than about 100:1. In some embodiments, the ratio of red blood cells to target cells is greater than about 250:1. In some embodiments, the ratio of red blood cells to target cells is greater than about 500:1. In some embodiments, the ratio of red blood cells to target cells is greater than about 1,000:1.

In some embodiments, the one or more target cells comprise peripheral blood mononuclear cells. In some embodiments, the one or more target cells comprise a stem cell, a lymphoid cell, or a myeloid cell. In some embodiments, the stem cell is a hematopoietic stem cell. In some embodiments, the lymphoid cell is a T cell. In some embodiments, the T cell displays a naïve phenotype. In some embodiments, the T cell displays a central memory phenotype. In some embodiments, the lymphoid cell is a natural killer cell or a natural killer T cell. In some embodiments, the myeloid cell is a dendritic cell. In some embodiments, the myeloid cell is a macrophage cell. In some embodiments, the one or more target cells comprise an exogenous nucleic acid encoding a chimeric antigen receptor or a recombinant T cell receptor. In some embodiments, the one or more target cells comprises an activated T cell. In some embodiments, the cell population is substantially free of one or more immune suppressive cells. In some embodiments, the immune suppressive cells are regulatory T cells. In some embodiments, the immune suppressive cells are regulatory B cells. In some embodiments, the immune suppressive cells comprise myeloid derived suppressor cells. In some embodiments, the one or more target cells possess the capacity to divide at least 3 time before exhaustion.

Disclosed are also processes for obtaining purified target cells from a blood related sample, wherein the blood related sample comprises target cells and red blood cells, the process comprising the steps of: (a) collecting the blood related sample from a patient; (b) removing platelets from the blood related sample collected in step (a); (c) optionally removing specific cells, other than platelets, from the sample prepared in step (b); (d) removing the red blood cells from the target cells after step b), or, if performed, after step (c) to obtain purified target cells; wherein, prior to step d, the red blood cell concentration in the blood related sample is maintained at, or adjusted to, at least 1×10⁴ red blood cells per microliter (μL).

In some embodiments, the patient is administered an anticoagulant for 1-10 days prior to the collection of the blood related sample.

In some embodiments, prior to step d), the red blood cell concentration in the blood related sample is maintained at, or adjusted to, a concentration of at least 1×10⁵ red blood cells per microliter (μL). In some embodiments, prior to step d), the red blood cell concentration in the blood related sample is maintained at, or adjusted to, a concentration of at least 5×10⁵ red blood cells per microliter (μL). In some embodiments, prior to step d), the red blood cell concentration in the blood related sample is maintained at, or adjusted to, a concentration of at least 1×10⁶ red blood cells per microliter (μL). In some embodiments, prior to step d), the red blood cell concentration in the blood related sample is maintained at, or adjusted to, a concentration of at least 5×10⁶ red blood cells per microliter (μL).

In some embodiments, the anticoagulant is added during the collection of blood in step a) using an in-line mixer. In some embodiments, the anticoagulant is a divalent metal chelator.

In some embodiments, the removal of platelets is initiated within 12 hours after the collection of blood is complete. In some embodiments, the removal of platelets is initiated within 6 hours after the collection of blood is complete. In some embodiments, the removal of platelets is initiated within 3 hours after the collection of blood is complete. In some embodiments, the removal of platelets is initiated within 1 hour after the collection of blood is complete. In some embodiments, the removal of platelets is initiated within 30 minutes after the collection of blood is complete. In some embodiments the primary objective is the removal of platelets rather that maintaining a high yield of target cells. In some embodiments, in step b), platelets are removed by size, density, electric charge, acoustic properties, or any combination of these parameters on a microfluidic device or series of devices.

In some embodiments, in step b), platelets are removed by Deterministic Lateral Displacement (DLD) on a microfluidic device, wherein the device comprises:

at least one channel extending from a sample inlet to one or more fluid outlets, wherein the channel is bounded by a first wall and a second wall opposite from the first wall;

an array of obstacles arranged in rows in the channel, each subsequent row of obstacles being shifted laterally with respect to a previous row, and wherein the obstacles are disposed in a manner such that, when the blood related sample is applied to an inlet of the device and fluidically passed through the channel, target cells flow to one or more collection outlets where an enriched product is collected and platelets flow to one more waste outlets that are separate from the collection outlets.

In some embodiments, in step d), red blood cells are removed by size; density; electric charge; acoustic properties or any combination of these parameters on a microfluidic device.

In some embodiments, in step d), red blood cells are removed from target cells by Deterministic Lateral Displacement (DLD) on a microfluidic device, wherein the device comprises:

at least one channel extending from a sample inlet to one or more fluid outlets, wherein the channel is bounded by a first wall and a second wall opposite from the first wall;

an array of obstacles arranged in rows in the channel, each subsequent row of obstacles being shifted laterally with respect to a previous row, and wherein the obstacles are disposed in a manner such that, when a sample comprising red blood cells and target cells is applied to an inlet of the device and fluidically passed through the channel, target cells flow to one or more collection outlets where an enriched product is collected and red blood cells flow to one more waste outlets that are separate from the collection outlets.

In some embodiments, after the purified target cells are obtained in step d) they are genetically engineered to have a desired phenotype. In some embodiments, after purified target cells are obtained or genetically engineered, they are expanded in culture. In some embodiments, after purified target cells are obtained or genetically engineered, they are used to treat the same patient from which the blood sample was obtained. In some embodiments, the target cells are leukocytes, stem cells, immune or hematopoietic cells. In some embodiments, the target cells are T cells.

Disclosed are processes for producing CAR T cells, comprising: (a) collecting a blood related sample comprising T cells from a patient; (b) removing platelets from the blood related sample collected in step a) (c) removing contaminant cells, other than platelets, from the sample prepared in step b); (d) removing the red blood cells from the T cells after step c) to obtain purified T cells; (e) genetically engineering the purified T cells to express the chimeric antigen receptors (CARs) on their surface, wherein, prior to step d), the red blood cell concentration in the blood related sample is maintained at, or adjusted to, at least 1×10⁴ red blood cells per microliter (μL).

In some embodiments, either before or after the purified T cells are genetically engineered, they are expanded in cell culture. In some embodiments, the purified T cells are combined with a T cell activator one to 1-5 days before being genetically engineered, but no activator is added to the T cells prior to that time. In some embodiments, the cells are activated for a period of 1-5 days before being genetically engineered. In some embodiments, the T cell activator is added within 24 hours after purified T cells are obtained. In some embodiments, the cells are genetically engineered by viral transformation wherein a viral vector is added to purified T cells either sequentially or simultaneously with a T cell activator, cells are washed after virus integration and then the transformed cells are immediately reinfused into the patient.

In some embodiments, the cells are genetically engineered by viral transformation wherein activator, a viral vector and growth factors are added to purified T cells in one step and the cells are cultured ex-vivo, for subsequent re-infusion.

In some embodiments, after culturing, cells are reinfused into the patient without being frozen. In some embodiments, after culturing, cells are frozen before being reinfused into the patient. In some embodiments, the T cell activator is a cytokine or antibody the activator may be used either in solution or immobilized on a bead or carrier. In some embodiments, the T cell activator is a magnetic bead coated with anti-CD3/CD28 antibodies. In some embodiments, the T cell activator is a T cell specific antibody or nanobead carrying a T cell specific antibody. In some embodiments, the T cell activator is a nano-matrix or soluble reagent that activate.

In some embodiments, naive T cells are isolated by immunoselective separation, non-naive T cells are removed by immunoselective separation and the naive T cells are activated either before separation (together with other T cells) or individually after immuno separation. In some embodiments, the T cell activator is removed from the T cells prior to genetic engineering. In some embodiments, the T cell activator is not removed from the T cells prior to genetic engineering. In some embodiments, the purified T cells are concentrated before being genetically engineered. In some embodiments, cells are concentrated by DLD on a microfluidic device.

In some embodiments, the CARs comprise a) an extracellular region comprising antigen binding domain; b) a transmembrane region; c) an intracellular region and wherein the CAR T cells optionally comprise one or more recombinant sequences that provide the cells with a molecular switch that, when triggered, reduce CAR T cell number or activity. In some embodiments, the T cells are derived from a patient with cancer, an autoimmune disease or an infectious disease. In some embodiments, in step c), T regulatory cells are removed. In some embodiments, the T regulatory cells are removed using CD 25 as a marker. In some embodiments, the T regulatory cells are removed using microbeads with antibodies recognizing CD 25 on their surface. In some embodiments, in step c), activated T cells are removed. In some embodiments, the activated cells are removed using CD69 or CD 25 as a marker.

In some embodiments, in step c), antigen presenting cells are removed.

In some embodiments, the antigen presenting cells are B cells. In some embodiments, the B cells are removed using CD19, CD10 or CD20 as a marker. In some embodiments, the B cells are removed using microbeads with antibodies recognizing CD19, CD10 or CD20 on their surface.

In some embodiments, in step c), dendritic cells are removed. In some embodiments, the dendritic cells are removed using CLEC9a, CD1c, CD11c, or CD141, CD14, CD205, CD83, BDCA1, or BDCA2 as a marker.

In some embodiments, in step c), granulocytes are removed. In some embodiments, the granulocytes are removed using CD16 and optionally CD66, and/or CD11b, as a marker.

In some embodiments, the patient is administered an anticoagulant for 1-10 days prior to the collection of the blood related sample.

In some embodiments, prior to step d), the red blood cell concentration in the blood related sample is maintained at, or adjusted to, a concentration of at least 1×10⁵ red blood cells per microliter (microliter (μL)). In some embodiments, prior to step d), the red blood cell concentration in the blood related sample is maintained at, or adjusted to, a concentration of at least 5×10⁵ red blood cells per microliter (microliter (μL)). In some embodiments, prior to step d), the red blood cell concentration in the blood related sample is maintained at, or adjusted to, a concentration of at least 1×10⁶ red blood cells per microliter (microliter (μL)). In some embodiments, prior to step d), the red blood cell concentration in the blood related sample is maintained at, or adjusted to, a concentration of at least 5×10⁶ red blood cells per microliter (microliter (μL)).

In some embodiments, anticoagulant is added during the collection of blood in step a) using an in-line mixer. In some embodiments, the anticoagulant is a divalent metal chelator.

In some embodiments, the removal of platelets is initiated within 12 hours after the collection of blood is complete. In some embodiments, he removal of platelets is initiated within 6 hours after the collection of blood is complete. In some embodiments, the removal of platelets is initiated within 3 hours after the collection of blood is complete. In some embodiments, the removal of platelets is initiated within 1 hour after the collection of blood is complete. In some embodiments, the removal of platelets is initiated within 30 minutes after the collection of blood is complete. In some embodiments, T cell activator is added within 24 hours after the collection of blood is complete. In some embodiments, in step b), platelets are removed by size, density, electric charge, acoustic properties, or any combination of these parameters on a microfluidic device or series of devices.

In some embodiments, in step b), platelets are removed by Deterministic Lateral Displacement (DLD) on a microfluidic device, wherein the device comprises: at least one channel extending from a sample inlet to one or more fluid outlets, wherein the channel is bounded by a first wall and a second wall opposite from the first wall; an array of obstacles arranged in rows in the channel, each subsequent row of obstacles being shifted laterally with respect to a previous row, and wherein the obstacles are disposed in a manner such that, when a sample comprising red blood cells and T cells is applied to an inlet of the device and fluidically passed through the channel T cells flow to one or more collection outlets where an enriched product is collected and platelets flow to one more waste outlets that are separate from the collection outlets.

In some embodiments, in step d), red blood cells are platelets are removed by size, density, electric charge, acoustic properties, or any combination of these parameters on a microfluidic device or series of devices.

In some embodiments, in step d), red blood cells are removed from target cells by Deterministic Lateral Displacement (DLD) on a microfluidic device, wherein the device comprises: at least one channel extending from a sample inlet to one or more fluid outlets, wherein the channel is bounded by a first wall and a second wall opposite from the first wall; an array of obstacles arranged in rows in the channel, each subsequent row of obstacles being shifted laterally with respect to a previous row, and wherein the obstacles are disposed in a manner such that, when a sample comprising red blood cells and T cells is applied to an inlet of the device and fluidically passed through the channel, T cells flow to one or more collection outlets where an enriched product is collected and red blood cells flow to one more waste outlets that are separate from the collection outlets.

In some embodiments, after T cells are genetically engineered in step e), T cells are separated from transformation agents and transferred into stabilization buffer, growth medium or cell culture medium.

In some embodiments, in step d), red blood cells are removed from target cells by Deterministic Lateral Displacement (DLD) on a microfluidic device, wherein the device comprises:

at least one channel extending from a sample inlet to one or more fluid outlets, wherein the channel is bounded by a first wall and a second wall opposite from the first wall;

an array of obstacles arranged in rows in the channel, each subsequent row of obstacles being shifted laterally with respect to a previous row, and wherein the obstacles are disposed in a manner such that, when a sample comprising transformation agents and T cells is applied to an inlet of the device and fluidically passed through the channel, T cells flow to one or more collection outlets where an enriched product is collected and transformation agents flow to one more waste outlets that are separate from the collection outlets.

In some embodiments, centrifugation is not performed during the process. In some embodiments, cells are not frozen at any point in the process.

Further disclosed are methods for obtaining target cells from a blood related sample, wherein the blood related sample comprises target cells, platelet cells, and red blood cells, the process comprising the steps of: (a) reducing platelets from the blood related sample, thereby providing a reduced platelet blood related sample; and (b) reducing or adjusting red blood cells of the reduced platelet blood related sample, thereby providing an adjusted red blood cell, reduced platelet blood related sample; wherein the adjusted red blood cell, reduced platelet blood related sample comprises at least about 1×10³ red blood cells per microliter to about 1×10⁷ per microliter.

In some embodiments, the methods comprise removing one or more non-target cells from the reduced platelet blood related sample or the adjusted red blood cell, reduced platelet blood related sample. In some embodiments, the non-target cells are selected from the list consisting of regulatory T cells, regulatory B cells, and granulocytes. In some embodiments, the non-target cells are regulatory T cells. In some embodiments, the non-target cells are regulatory B cells. In some embodiments, the non-target cells are granulocytes.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A-B illustrates, in part, the advantages conferred by the methods and compositions disclosed herein.

FIGS. 2A-B shows, in part, orders for processing a sample comprising target cells.

FIG. 3 illustrates the experimental design utilized in the examples.

FIG. 4 shows data demonstrating the effect of plasma and red blood cells on stimulation response as measured by CD4 conversion to CD8 T cell phenotype (CD3/CD28 stimulation +IL7/15).

FIGS. 5A-B shows data demonstrating the effect of plasma/platelet removal and red blood cells on CD4, CD8 Memory-Naïve Balance prior to CD3/CD28 co-stimulation and isolation into culture at Day 6.

FIG. 6 shows the effect of plasma/platelet removal and hematocrit on total T cell expansion prior to CD3/CD28 costimulation and isolation into culture.

### DETAILED DESCRIPTION

Cell-based therapies generally require the isolation of a specific type of cell or cell population (i.e. targets cells) that are, in turn, used for a therapeutic purpose. In some cell-based therapies, the collected target cells are further engineered (e.g. genetically modified) to generate therapeutic potential. Consequently, the processes and methods used to collect or isolate target cells, and intermediates therein, play an important role in the generation of the therapeutic cell product (i.e. therapeutically active cells). During the collection and isolation process, the target cells are subject to, and respond to, factors (e.g. soluble factors, platelets, non-target cells, etc.) within the environment that surrounds the target cells. As provided herein, the effects of compositions, methods, and systems utilized in the isolation and processing of target cells play a role in determining the quantity and quality of collected target cells use for the generation of a therapeutic cell product for the treatment of disease.

The quantity of collected target cells is not the only determinant of success in the generation of therapeutic cell products. Characteristics of collected target cells (e.g. peripheral blood mononuclear cells, leukocytes, immune cells, etc.), beyond cell quantity, can also affect the ability to successfully manufacture an effective therapeutic cell product. During the isolation, collection, and/or purification process, target cells and cells within a sample are subject to an array of physical (e.g. mechanical forces, etc.), chemical (e.g. compounds, substances, etc.), and biological (e.g. soluble factors, platelets, non-target cells, etc.) interactions. The interplay of such interactions ultimately shapes the collected cells (or collected cell product) used for the generation of the therapeutic cell product. Disclosed are compositions, methods, and systems that use the ordered processing of a sample containing cells to shape the of physical, chemical, and biological interactions that a target cell is subjected to during collection in order to provide collected targets cells capable of generating effective cell-based therapies.

The compositions, methods, and systems disclosed herein provide advantages for improving the quantity and quality collected target cells (e.g. the collected cell product) from a blood sample. One problem with the use of blood samples as a source of therapeutic cells is that the physical (e.g. mechanical forces, etc.), chemical (e.g. compounds, substances, etc.), and biological (e.g. soluble factors, platelets, non-target cells, etc.) interactions resulting from the methods and processes used in collection of blood can negatively impact the usability of certain collected target cells or cell populations for use in generating a therapeutic cell product. For example, such methods and processes can initiate biological pathways (e.g. cellular signaling cascades) that prevent or decrease the usability of collected target cells in the generation of cell therapies. Methods and processes for collecting target cells from blood (e.g. apheresis) generally comprise perturbations that affect the properties and activity of the collected target cells. For example, in addition to the classical coagulation cascade following intrinsic or extrinsic platelet activation, changes in hemodynamic balance also initiate cellular responses that can prevent or decrease the usability of collected target cells from blood. Additionally, excess contact, perturbation and resultant cell signaling has been documented to induce cell activation, anergy and even tonic signaling (increased frequency of T cell:B cell interactions) within the sample. Thus, collected target cells for therapy are changed by the act of collection and processing in ways that affect their ability to respond to downstream engineering processes for use in the generation of therapeutically active cells.

The compositions, methods, and systems provided herein address the challenges and problems associated with methods and processes for isolating target cells for use in generating therapeutically active cells. The compositions, methods, and systems provided are based on the discovery that maintaining the presence of erythrocytes (i.e. red blood cells) in a sample of target cells increases the quantity and quality of collected target cells or increases the quantity and quality of collected target cells in a desired state. For example, maintaining a ratio of red blood cells (RBCs) to other cells (e.g. target cells or cell populations comprising target cells) in a sample by ordered processing of cells within the sample, and/or adding red blood cells (RBCs) to a composition of target cells, increases the quantity and/or quality of collected target cells recovered from a blood sample. As another example, the removal of platelets while maintaining a presence of red blood cells (RBCs) in a sample can also increase the quantity and/or quality of collected target cells. Without being bound by theory, the improved quantity can be attributed to effect of red blood cells (RBCs) in increasing target cell viability by shaping the physical (e.g. mechanical forces, etc.), chemical (e.g. compounds, substances, etc.), and biological (e.g. soluble factors, platelets, non-target cells, etc.) interactions.

The disclosure herein substantially facilitates the generation of the isolation of target cells for the development and manufacture of cell therapies. Maintaining and/or controlling the presence of red blood cells (RBCs) in a sample of target cells increases the quantity and quality of collected target cells used for the generation of cell therapies. In short, the better starting material (i.e. collected target cells) results in a better product (e.g. a therapeutic cell product). For example, T cells for engineered T cell (e.g. chimeric antigen receptor T cell (CAR-T), modified T-cell receptor T cells (TCR-T), etc.) based therapies are generally collected by leukapheresis wherein leukocytes (i.e. white blood cells) are separated from both platelets and white blood cells by, for example, centrifugation whereas, the compositions and methods disclosed herein maintain a presence of red blood cells (RBCs) with the leukocytes. Here, the presence of red blood cells increases the viability of the broader genus of target cells (i.e. leukocytes) and increases the viability and expansion capacities of T cells for engineering of a chimeric antigen receptor T cell (CAR-T) product. Such compositions and methods may further comprise the absence or removal non-target cells (e.g. leukocytes that are not T cells) at any point during processing.

Furthermore, practice of the disclosure provides a solution to clinically relevant challenges relative to stratifying patients suitable for cell therapies based on a threshold number of target cells collected from a patient. For example, most clinical applications require a baseline number of absolute lymphocyte counts (ALCs), wherein the ALC value is directly tied to the collection methods and viability and/or expansion capacities of the target cells generated therefrom. Therefore, increasing the viability and/or expansion capacities of collected target cells (e.g. the collected cell product) by maintaining and/or controlling the presence of red blood cells (RBCs) in a sample of target cells can increase the number of patients eligible for cell therapies by lowering a baseline value of targets cells needed for generating a therapeutic cell product. Accordingly, application of the disclosure enables the generation of improved and/or superior collected cell products that, in turn, provide advantages and solutions for the challenges associated with the manufacture of cell therapies.

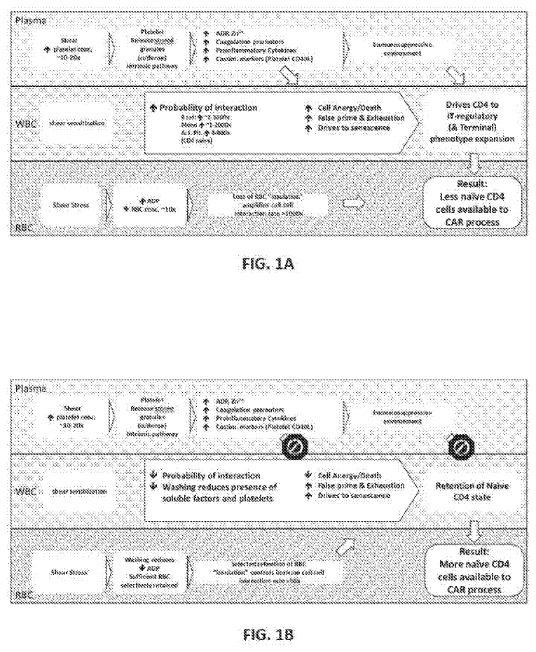

Canonical processing of T cells (e.g. target cells) for therapeutic use comprises the isolation of white blood cells (WBC) from both plasma and red blood cells. As exemplified in FIG. 1A, the removal of red blood cells not only results in reduced protection from the shear stress forces, but also results in the increased cell to cell interactions that drive reduced viability and expansion capacities (e.g. less naïve CD4 cells are available for CAR engineering) of the T cells (e.g. target cells). Furthermore, the presence of platelets in a sample subject the T cells (e.g. target cells) to factors that drive reduced viability and expansion capacities (e.g. immunosuppressive factors). FIG. 1B exemplifies the solution and benefits to target cell processing methods and target cell compositions that comprise red blood cells. The presence of red blood cells insulates or cushions target cells from cell to cell interactions that drive reduced viability and expansion capacities (e.g. less naïve CD4 cells are available for CAR engineering) of the T cells (e.g. target cells). Such principles are exemplified and disclosed herein. Accordingly, the disclosed provides compositions and methods that confer solutions and advantages to the challenges associated with isolating target cells for use in the development of therapeutic cells.

Described herein, in one aspect, is a method for processing a blood related sample comprising: (a) providing a blood related sample comprising one or more target cells, platelet cells, red blood cells; and (b) reducing a number of the platelet cells in the blood related sample while maintaining or adjusting a ratio of the red blood cells to the one or more target cells greater than about 50:1 to produce a reduced platelet blood related sample comprising the one or more target cells.

Described herein, in another aspect, is a cell population comprising one or more target cells, platelet cells and red blood cells, the target cells at a ratio of platelets to target cells less than about 500:1 and at a ratio of red blood cells to target cells of greater than about 50:1.

### Cell Population Compositions for Improved Cell Therapy Manufacture

The advantages and solutions for providing improved or superior collected cell products (e.g. cell populations) is based on the phenomena that a presence of erythrocytes (i.e. red blood cells) in a sample or composition of target cells improves the quantity and quality of the target cells used for generating cell therapies. Generally, advantages are achieved by maintaining a presence of red blood cells (RBCs) throughout the collection or isolation process. The advantages can also be conferred by the presence of the red blood cells (RBCs) in the processing steps comprising the expansion of target cells and/or the genetic engineering/modification of the target cells (e.g. viral transduction, transfection, gene editing, etc.).

The compositions disclosed herein generally comprise target cells and red blood cells, and are useful for providing collected target cell products having improved quantitative and qualitative yields. The improved quantitative and qualitative yields can refer to the number or properties of targets cells prior to expansion of the target cells or the quantitative and qualitative yields obtained after expansion of target cells. For example, a collected target cell product (e.g. leukocytes) comprising red blood cells (RBCs) and target cells (e.g. T cells) can produce an increased number of expanded target cells (e.g. T cells) as compared to a collected target cell composition not having red blood cells. As another example, a fewer number of target cells from collected target cell sample comprising red blood cells (RBCs) and target cells may be required for the generating a expanded target cell population sufficient for generating a therapeutic cell product as compared to collected target cell composition not comprising red blood cells (RBCs). Cells or target cells that are used therapeutically are often developed in stages that can take place either in vivo or in vitro. For example, in response to an antigen presented by an activated antigen presenting cell in vivo or stimulation with anti-CD3 and anti-CD28 antibody in vitro, naive T cells begin a process in which they develop into T memory stem cells, followed by central memory T cells, effector memory cells and finally short lived effector T cells (see Gattinoni, et al.; Moving T memory stem cells to the clinic, Blood 121(4):567-568 (2013)). Factors known to be capable of affecting this process include soluble factors such as IL-7, IL-15 and TWS119 (promoting the progression of naïve T cells to T memory stem cells) and IL-2 (promoting the development of naive T cells into effector memory cells (Id.), and cell-cell interactions (e.g., interaction of costimulatory molecules on antigen presenting cells with T cells).

Cells often must also be engineered to realize their full potential as therapeutic agents. This may take the form, for example, of promoting the targeting of a specific cell type or altering a genetic lesion. In virtually all cases, the ability of the cell to divide and successfully integrate a genetic insert is fundamental and, to maintain other therapeutically valuable attributes, is critical to the yield of active cells. By way of example, CAR-T cell manufacture introduces a specific affinity targeting construct or constructs. Further, the type of T cell that is preferred as a therapeutic construct is a T memory cell and preferably a T memory stem cell. Obtaining a high yield of these cells will depend on both eliminating factors that may be present in cell preparations that steer cells to unwanted ends and adding factors that steer the cells to their most therapeutically desirable state. It should also be recognized that, as the number of T cell doublings increases, the proportion of less desirable effector cells increases. Therefore, controlling the number of doublings is important.

Accordingly, compositions comprising an effective ratio of red blood cells (RBCs) to target cells are useful for generating a collected target cell product (e.g. a cell population) having improved quantitative and qualitative properties for producing a cell therapy. In some embodiments, the ratio of red blood cells (RBCs) to target cells is about 1:1 to about 1,000:1. In some embodiments, the ratio of red blood cells (RBCs) to target cells is about 1:1 to about 10:1, about 1:1 to about 50:1, about 1:1 to about 100:1, about 1:1 to about 200:1, about 1:1 to about 300:1, about 1:1 to about 400:1, about 1:1 to about 500:1, about 1:1 to about 600:1, about 1:1 to about 700:1, about 1:1 to about 800:1, about 1:1 to about 1,000:1, about 10:1 to about 50:1, about 10:1 to about 100:1, about 10:1 to about 200:1, about 10:1 to about 300:1, about 10:1 to about 400:1, about 10:1 to about 500:1, about 10:1 to about 600:1, about 10:1 to about 700:1, about 10:1 to about 800:1, about 10:1 to about 1,000:1, about 50:1 to about 100:1, about 50:1 to about 200:1, about 50:1 to about 300:1, about 50:1 to about 400:1, about 50:1 to about 500:1, about 50:1 to about 600:1, about 50:1 to about 700:1, about 50:1 to about 800:1, about 50:1 to about 1,000:1, about 100:1 to about 200:1, about 100:1 to about 300:1, about 100:1 to about 400:1, about 100:1 to about 500:1, about 100:1 to about 600:1, about 100:1 to about 700:1, about 100:1 to about 800:1, about 100:1 to about 1,000:1, about 200:1 to about 300:1, about 200:1 to about 400:1, about 200:1 to about 500:1, about 200:1 to about 600:1, about 200:1 to about 700:1, about 200:1 to about 800:1, about 200:1 to about 1,000:1, about 300:1 to about 400:1, about 300:1 to about 500:1, about 300:1 to about 600:1, about 300:1 to about 700:1, about 300:1 to about 800:1, about 300:1 to about 1,000:1, about 400:1 to about 500:1, about 400:1 to about 600:1, about 400:1 to about 700:1, about 400:1 to about 800:1, about 400:1 to about 1,000:1, about 500:1 to about 600:1, about 500:1 to about 700:1, about 500:1 to about 800:1, about 500:1 to about 1,000:1, about 600:1 to about 700:1, about 600:1 to about 800:1, about 600:1 to about 1,000:1, about 700:1 to about 800:1, about 700:1 to about 1,000:1, or about 800:1 to about 1,000:1. In some embodiments, the ratio of red blood cells (RBCs) to target cells is about 1:1, about 10:1, about 50:1, about 100:1, about 200:1, about 300:1, about 400:1, about 500:1, about 600:1, about 700:1, about 800:1, or about 1,000:1. In some embodiments, the ratio of red blood cells (RBCs) to target cells is at least about 1:1, about 10:1, about 50:1, about 100:1, about 200:1, about 300:1, about 400:1, about 500:1, about 600:1, about 700:1, about 800:1, about 900:1, or about 1,000:1.

Red blood cells (RBCs) within a sample or collected cell product (e.g. cell population) can be defined by the volume fraction of a sample (e.g. cell population) occupied by erythrocytes (i.e. red blood cells), also known as hematocrit. The hematocrit (hct) is expressed as a percentage. For example, a hematocrit of 10% means that there are 10 milliliters of red blood cells in 100 milliliters of blood. Compositions comprising an effective hematocrit percentage of red blood cells (RBCs) relative to target cells are useful for generating a collected target cell product (e.g. a cell population) having improved quantitative and qualitative properties for producing a cell therapy.

In some embodiments, the hematocrit percentage of red blood cells (RBCs) in a sample consisting of target cells and red blood cells (RBCs) is about 0.5% to about 50%. In some embodiments, the hematocrit percentage of red blood cells (RBCs) in a sample consisting of target cells and red blood cells (RBCs) is about 0.5% to about 1%, about 0.5% to about 5%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.5% to about 45%, about 0.5% to about 50%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the hematocrit percentage of red blood cells (RBCs) in a sample consisting of target cells and red blood cells (RBCs) is about 0.5%, about 1%, about 5%, about 10%, about 15%, about 20%, about 25%, about 30%, about 35%, about 40%, about 45%, or about 50%. In some embodiments, the hematocrit percentage of red blood cells (RBCs) in a sample consisting of target cells and red blood cells (RBCs) is at least about 0.5%, about 1%, about 5%, about 10%, about 15%, about 20%, about 25%, about 30%, about 35%, about 40%, or about 45%.

Compositions comprising an effective ratio of red blood cells (RBCs) to total cells (target and non-target cells) of the composition are useful for generating a cell population comprising target cells and having improved quantitative and qualitative properties for producing a cell therapy. Accordingly, compositions comprising an effective ratio of red blood cells (RBCs) to target cells are useful for generating a collected target cell product (e.g. a cell population) having improved quantitative and qualitative properties for producing a cell therapy. In some embodiments, the ratio of red blood cells (RBCs) to total cells is about 1:1 to about 1,000:1. In some embodiments, the ratio of red blood cells (RBCs) to total cells is about 1:1 to about 10:1, about 1:1 to about 50:1, about 1:1 to about 100:1, about 1:1 to about 200:1, about 1:1 to about 300:1, about 1:1 to about 400:1, about 1:1 to about 500:1, about 1:1 to about 600:1, about 1:1 to about 700:1, about 1:1 to about 800:1, about 1:1 to about 1,000:1, about 10:1 to about 50:1, about 10:1 to about 100:1, about 10:1 to about 200:1, about 10:1 to about 300:1, about 10:1 to about 400:1, about 10:1 to about 500:1, about 10:1 to about 600:1, about 10:1 to about 700:1, about 10:1 to about 800:1, about 10:1 to about 1,000:1, about 50:1 to about 100:1, about 50:1 to about 200:1, about 50:1 to about 300:1, about 50:1 to about 400:1, about 50:1 to about 500:1, about 50:1 to about 600:1, about 50:1 to about 700:1, about 50:1 to about 800:1, about 50:1 to about 1,000:1, about 100:1 to about 200:1, about 100:1 to about 300:1, about 100:1 to about 400:1, about 100:1 to about 500:1, about 100:1 to about 600:1, about 100:1 to about 700:1, about 100:1 to about 800:1, about 100:1 to about 1,000:1, about 200:1 to about 300:1, about 200:1 to about 400:1, about 200:1 to about 500:1, about 200:1 to about 600:1, about 200:1 to about 700:1, about 200:1 to about 800:1, about 200:1 to about 1,000:1, about 300:1 to about 400:1, about 300:1 to about 500:1, about 300:1 to about 600:1, about 300:1 to about 700:1, about 300:1 to about 800:1, about 300:1 to about 1,000:1, about 400:1 to about 500:1, about 400:1 to about 600:1, about 400:1 to about 700:1, about 400:1 to about 800:1, about 400:1 to about 1,000:1, about 500:1 to about 600:1, about 500:1 to about 700:1, about 500:1 to about 800:1, about 500:1 to about 1,000:1, about 600:1 to about 700:1, about 600:1 to about 800:1, about 600:1 to about 1,000:1, about 700:1 to about 800:1, about 700:1 to about 1,000:1, or about 800:1 to about 1,000:1. In some embodiments, the ratio of red blood cells (RBCs) to total cells is about 1:1, about 10:1, about 50:1, about 100:1, about 200:1, about 300:1, about 400:1, about 500:1, about 600:1, about 700:1, about 800:1, or about 1,000:1. In some embodiments, the ratio of red blood cells (RBCs) to total cells is at least about 1:1, about 10:1, about 50:1, about 100:1, about 200:1, about 300:1, about 400:1, about 500:1, about 600:1, about 700:1, or about 800:1.

In some embodiments, the hematocrit percentage of red blood cells (RBCs) in a sample consisting of target cells, non-target cells, and red blood cells (RBCs) is about 0.5% to about 50%. In some embodiments, the hematocrit percentage of red blood cells (RBCs) in a sample consisting of target cells, non-target cells, and red blood cells (RBCs) is about 0.5% to about 1%, about 0.5% to about 5%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.5% to about 45%, about 0.5% to about 50%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the hematocrit percentage of red blood cells (RBCs) in a sample consisting of target cells, non-target cells, and red blood cells (RBCs) is about 0.5%, about 1%, about 5%, about 10%, about 15%, about 20%, about 25%, about 30%, about 35%, about 40%, about 45%, or about 50%. In some embodiments, the hematocrit percentage of red blood cells (RBCs) in a sample consisting of target cells, non-target cells, and red blood cells (RBCs) is at least about 0.5%, about 1%, about 5%, about 10%, about 15%, about 20%, about 25%, about 30%, about 35%, about 40%, or about 45%.

Compositions having a limited the quantity of platelets or reduced number of platelets while maintaining a presence of red blood cells (RBCs) are also beneficial for generating compositions of collected cell products (e.g. cell populations) comprising red blood cells (RBCs) and target cells. The compositions are useful for generating a collected target cell product (e.g. a cell population) having improved quantitative and qualitative properties for producing a cell therapy. Platelets contribute to inflammatory reactions in vivo through the release of soluble pro-inflammatory proteins. In vitro or during sample processing these pro-inflammatory mediators can be released and have pleiotropic effects on different lymphocyte populations. Therefore, methods described herein provide for removing or reducing the number of platelets or thrombocytes from a blood-related sample. According to the methods provided herein platelets can be removed prior to removal of red blood cells, and/or other non-target cells. The removal of platelets generates a reduced platelet blood related sample. Platelets can be removed by any suitable method including the use of platelet pheresis microfluidic cell-sorting methods, affinity purification, or appropriate density centrifugation. In certain embodiments, platelets are removed before removal or reduction of red blood cells. In certain embodiments, platelets are removed before removal or reduction of non-target cells.

The normal physiological concentration of platelets in whole-blood is between 150,000 to 300,000 per microliter. Therefore, the concentration of platelets in the platelet reduced collected target cell product is decreased as compared to the input. Furthermore, the input may be any composition comprising target cells (e.g. a leukopack, a leukaphereis product, an apheresis product, etc.). Accordingly, further reduction of platelets below the initial input or starting sample is desirable. As another example, the concentration of platelets in the reduced platelet target cell product comprising, at least, target cells and red blood cells (RBCs) is less than about 15,000, 10,000, 5,000, 2,500, 1,500, 1,000, 500, 100, or 50 platelets per microliter. These numbers coincide with a reduction of platelets by 90% to 99.97% compared to a starting blood related sample. While reduction of platelet concentration in the reduced platelet related blood sample by the method described herein may result in a residual or trace amounts of platelets such as 1, 5, 10, 50, or 100 platelets per microliter in the reduced platelet blood related sample.

Accordingly, platelet-reduced compositions comprising an effective ratio of platelets to target cells are useful in combination with the effective ratio of red blood cells (RBCs) to target cells or total cells, for generating a collected target cell product (e.g. a cell population) having improved quantitative and qualitative properties for producing a cell therapy. In some embodiments, in addition to the disclosed ratio of red blood cells (RBCs) to target cells or total cells, the ratio of platelets to target cells less than about 500:1. In some embodiments, in addition to the disclosed ratio of red blood cells (RBCs) to target cells or total cells, the ratio of platelets to target cells about 0.001:1 to about 0.01:1, about 0.001:1 to about 0.1:1, about 0.001:1 to about 1:1, about 0.001:1 to about 10:1, about 0.001:1 to about 25:1, about 0.001:1 to about 50:1, about 0.001:1 to about 100:1, about 0.001:1 to about 200:1, about 0.001:1 to about 300:1, about 0.001:1 to about 400:1, about 0.001:1 to about 500:1, about 0.01:1 to about 0.1:1, about 0.01:1 to about 1:1, about 0.01:1 to about 10:1, about 0.01:1 to about 25:1, about 0.01:1 to about 50:1, about 0.01:1 to about 100:1, about 0.01:1 to about 200:1, about 0.01:1 to about 300:1, about 0.01:1 to about 400:1, about 0.01:1 to about 500:1, about 0.1:1 to about 1:1, about 0.1:1 to about 10:1, about 0.1:1 to about 25:1, about 0.1:1 to about 50:1, about 0.1:1 to about 100:1, about 0.1:1 to about 200:1, about 0.1:1 to about 300:1, about 0.1:1 to about 400:1, about 0.1:1 to about 500:1, about 1:1 to about 10:1, about 1:1 to about 25:1, about 1:1 to about 50:1, about 1:1 to about 100:1, about 1:1 to about 200:1, about 1:1 to about 300:1, about 1:1 to about 400:1, about 1:1 to about 500:1, about 10:1 to about 25:1, about 10:1 to about 50:1, about 10:1 to about 100:1, about 10:1 to about 200:1, about 10:1 to about 300:1, about 10:1 to about 400:1, about 10:1 to about 500:1, about 25:1 to about 50:1, about 25:1 to about 100:1, about 25:1 to about 200:1, about 25:1 to about 300:1, about 25:1 to about 400:1, about 25:1 to about 500:1, about 50:1 to about 100:1, about 50:1 to about 200:1, about 50:1 to about 300:1, about 50:1 to about 400:1, about 50:1 to about 500:1, about 100:1 to about 200:1, about 100:1 to about 300:1, about 100:1 to about 400:1, about 100:1 to about 500:1, about 200:1 to about 300:1, about 200:1 to about 400:1, about 200:1 to about 500:1, about 300:1 to about 400:1, about 300:1 to about 500:1, or about 400:1 to about 500:1. In some embodiments, in addition to the disclosed ratio of red blood cells (RBCs) to target cells or total cells, the ratio of platelets to target cells about 0.001:1, about 0.01:1, about 0.1:1, about 1:1, about 10:1, about 25:1, about 50:1, about 100:1, about 200:1, about 300:1, about 400:1, or about 500:1. In some embodiments, in addition to the disclosed ratio of red blood cells (RBCs) to target cells or total cells, the ratio of platelets to target cells at most about 0.01:1, about 0.1:1, about 1:1, about 10:1, about 25:1, about 50:1, about 100:1, about 200:1, about 300:1, about 400:1, or about 500:1.

Platelet-reduced compositions comprising an effective ratio of platelets to target cells are useful in combination with the effective hematocrit percentage of red blood cells (RBCs) in a cell population, for generating a collected target cell product (e.g. a cell population) having improved quantitative and qualitative properties for producing a cell therapy. In some embodiments, in addition to the disclosed effective hematocrit percentage of red blood cells (RBCs) in a sample, the ratio of platelets to target cells less than about 500:1. In some embodiments, in addition to the disclosed effective hematocrit percentage of red blood cells (RBCs) in a sample, the ratio of platelets to target cells about 0.001:1 to about 0.01:1, about 0.001:1 to about 0.1:1, about 0.001:1 to about 1:1, about 0.001:1 to about 10:1, about 0.001:1 to about 25:1, about 0.001:1 to about 50:1, about 0.001:1 to about 100:1, about 0.001:1 to about 200:1, about 0.001:1 to about 300:1, about 0.001:1 to about 400:1, about 0.001:1 to about 500:1, about 0.01:1 to about 0.1:1, about 0.01:1 to about 1:1, about 0.01:1 to about 10:1, about 0.01:1 to about 25:1, about 0.01:1 to about 50:1, about 0.01:1 to about 100:1, about 0.01:1 to about 200:1, about 0.01:1 to about 300:1, about 0.01:1 to about 400:1, about 0.01:1 to about 500:1, about 0.1:1 to about 1:1, about 0.1:1 to about 10:1, about 0.1:1 to about 25:1, about 0.1:1 to about 50:1, about 0.1:1 to about 100:1, about 0.1:1 to about 200:1, about 0.1:1 to about 300:1, about 0.1:1 to about 400:1, about 0.1:1 to about 500:1, about 1:1 to about 10:1, about 1:1 to about 25:1, about 1:1 to about 50:1, about 1:1 to about 100:1, about 1:1 to about 200:1, about 1:1 to about 300:1, about 1:1 to about 400:1, about 1:1 to about 500:1, about 10:1 to about 25:1, about 10:1 to about 50:1, about 10:1 to about 100:1, about 10:1 to about 200:1, about 10:1 to about 300:1, about 10:1 to about 400:1, about 10:1 to about 500:1, about 25:1 to about 50:1, about 25:1 to about 100:1, about 25:1 to about 200:1, about 25:1 to about 300:1, about 25:1 to about 400:1, about 25:1 to about 500:1, about 50:1 to about 100:1, about 50:1 to about 200:1, about 50:1 to about 300:1, about 50:1 to about 400:1, about 50:1 to about 500:1, about 100:1 to about 200:1, about 100:1 to about 300:1, about 100:1 to about 400:1, about 100:1 to about 500:1, about 200:1 to about 300:1, about 200:1 to about 400:1, about 200:1 to about 500:1, about 300:1 to about 400:1, about 300:1 to about 500:1, or about 400:1 to about 500:1. In some embodiments, in addition to the disclosed effective hematocrit percentage of red blood cells (RBCs) in a sample, the ratio of platelets to target cells about 0.001:1, about 0.01:1, about 0.1:1, about 1:1, about 10:1, about 25:1, about 50:1, about 100:1, about 200:1, about 300:1, about 400:1, or about 500:1. In some embodiments, in addition to the disclosed effective hematocrit percentage of red blood cells (RBCs) in a sample, the ratio of platelets to target cells at most about 0.01:1, about 0.1:1, about 1:1, about 10:1, about 25:1, about 50:1, about 100:1, about 200:1, about 300:1, about 400:1, or about 500:1.

### Methods of Generating Cell Populations for Improved Cell Therapy Manufacture

Methods for generating collected target cell products (e.g. cell populations) that comprise target cells and erythrocytes are also useful for improving the quantitative and qualitative properties of the target cell(s) for producing a therapeutic cell product. The disclosure herein provides methods of purifying, collecting, or isolating target cells from blood-related samples for cell therapy applications. Such applications include without limitation stem cell manipulation, genetic engineering of cells, activating cells, and/or transplant. Appropriate stem cells for manipulation or transplantation include hematopoietic stem cells or other pluripotent cells that originate from bone marrow. The provision of cells that are manipulated or rendered transgenic for therapeutic applications is another use of the cells isolated or purified herein. Such cells can comprise peripheral blood mononuclear cells, including immune cells, such as T cells, B-cells, dendritic cells, macrophages, natural killer cells or natural killer T cells.

Blood-related samples for use according to the methods described herein include any sample comprising platelets, red blood cells, and one or more additional cell types. Blood-related samples can be whole-blood samples derived from one or more donors. Additional, samples can be those that have been previously subjected to partial or complete apheresis procedures, such as plasmapheresis, plateletpheresis, erythrocytapheresis, or leukapheresis. In certain embodiments, the blood related sample is an apheresis product. In certain embodiments, the blood related sample is a leukapheresis product. The blood related sample may comprise a volume in excess of about 1 milliliter, about 2 milliliters, about 5 milliliters, about 10 milliliters, about 25 milliliters, about 50 milliliters, about 100 milliliters, 250 milliliters, 500 milliliters, or more.

The disclosed methods provide for the generation of target cell populations or cell populations comprising target cells having improved quantitative and qualitative properties. Generating target cell populations or cell populations comprising target cells having improved quantitative and qualitative properties can be achieved by methods that (1) maintain a presence of red blood cells in a composition comprising target cells, or (2) limiting or reducing the number of platelets while maintaining a presence of red blood cells in a composition comprising target cells. Accordingly, disclosed are methods for processing a blood related sample comprising: (a) providing a blood-related sample comprising one or more target cells, platelets, and red blood cells, and (b) reducing a number of the platelet in the blood related sample while maintaining a ratio of the red blood cells to the one or more target cells greater than about 50:1 to produce a reduced platelet blood related sample comprising the one or more target cells. Maintaining the ratio of red blood cells to the one or more target cells can be achieved by the methods or systems employed for the processing a blood related sample. Maintaining the ratio of red blood cells to the one or more target cells can also be achieved adjusting ratio of red blood cells in a collected target cell product (e.g. cell population). For example, red blood cells can be added to collected leukocytes cells to maintain a ratio of red blood cells to the one or more target cells greater than about 50:1.

Methods for maintaining an effective ratio of red blood cells (RBCs) to target cells are useful for generating a collected target cell product (e.g. a cell population) having improved quantitative and qualitative properties for producing a cell therapy. In some embodiments, the method yields a ratio of red blood cells (RBCs) to target cells of about 1:1 to about 1,000:1. In some embodiments, the method yields a ratio of red blood cells (RBCs) to target cells of about 1:1 to about 10:1, about 1:1 to about 50:1, about 1:1 to about 100:1, about 1:1 to about 200:1, about 1:1 to about 300:1, about 1:1 to about 400:1, about 1:1 to about 500:1, about 1:1 to about 600:1, about 1:1 to about 700:1, about 1:1 to about 800:1, about 1:1 to about 1,000:1, about 10:1 to about 50:1, about 10:1 to about 100:1, about 10:1 to about 200:1, about 10:1 to about 300:1, about 10:1 to about 400:1, about 10:1 to about 500:1, about 10:1 to about 600:1, about 10:1 to about 700:1, about 10:1 to about 800:1, about 10:1 to about 1,000:1, about 50:1 to about 100:1, about 50:1 to about 200:1, about 50:1 to about 300:1, about 50:1 to about 400:1, about 50:1 to about 500:1, about 50:1 to about 600:1, about 50:1 to about 700:1, about 50:1 to about 800:1, about 50:1 to about 1,000:1, about 100:1 to about 200:1, about 100:1 to about 300:1, about 100:1 to about 400:1, about 100:1 to about 500:1, about 100:1 to about 600:1, about 100:1 to about 700:1, about 100:1 to about 800:1, about 100:1 to about 1,000:1, about 200:1 to about 300:1, about 200:1 to about 400:1, about 200:1 to about 500:1, about 200:1 to about 600:1, about 200:1 to about 700:1, about 200:1 to about 800:1, about 200:1 to about 1,000:1, about 300:1 to about 400:1, about 300:1 to about 500:1, about 300:1 to about 600:1, about 300:1 to about 700:1, about 300:1 to about 800:1, about 300:1 to about 1,000:1, about 400:1 to about 500:1, about 400:1 to about 600:1, about 400:1 to about 700:1, about 400:1 to about 800:1, about 400:1 to about 1,000:1, about 500:1 to about 600:1, about 500:1 to about 700:1, about 500:1 to about 800:1, about 500:1 to about 1,000:1, about 600:1 to about 700:1, about 600:1 to about 800:1, about 600:1 to about 1,000:1, about 700:1 to about 800:1, about 700:1 to about 1,000:1, or about 800:1 to about 1,000:1. In some embodiments, the method yields a ratio of red blood cells (RBCs) to target cells of about 1:1, about 10:1, about 50:1, about 100:1, about 200:1, about 300:1, about 400:1, about 500:1, about 600:1, about 700:1, about 800:1, or about 1,000:1. In some embodiments, the ratio of red blood cells (RBCs) to target cells is at least about 1:1, about 10:1, about 50:1, about 100:1, about 200:1, about 300:1, about 400:1, about 500:1, about 600:1, about 700:1, about 800:1, or about 900:1. In some embodiments, maintaining an effective ratio of red blood cells (RBCs) to target cells can be achieved by adding red blood cells to a composition of target cells to achieved or produce the effective ratio.

In some embodiments, the methods yield a hematocrit percentage of red blood cells (RBCs) in a sample consisting of target cells and red blood cells (RBCs) of about 0.5% to about 50%. In some embodiments, the methods yield a hematocrit percentage of red blood cells (RBCs) in a sample consisting of target cells and red blood cells (RBCs) of about 0.5% to about 1%, about 0.5% to about 5%, about 0.5% to about 10%, about 0.5% to about 15%, about 0.5% to about 20%, about 0.5% to about 25%, about 0.5% to about 30%, about 0.5% to about 35%, about 0.5% to about 40%, about 0.5% to about 45%, about 0.5% to about 50%, about 1% to about 5%, about 1% to about 10%, about 1% to about 15%, about 1% to about 20%, about 1% to about 25%, about 1% to about 30%, about 1% to about 35%, about 1% to about 40%, about 1% to about 45%, about 1% to about 50%, about 5% to about 10%, about 5% to about 15%, about 5% to about 20%, about 5% to about 25%, about 5% to about 30%, about 5% to about 35%, about 5% to about 40%, about 5% to about 45%, about 5% to about 50%, about 10% to about 15%, about 10% to about 20%, about 10% to about 25%, about 10% to about 30%, about 10% to about 35%, about 10% to about 40%, about 10% to about 45%, about 10% to about 50%, about 15% to about 20%, about 15% to about 25%, about 15% to about 30%, about 15% to about 35%, about 15% to about 40%, about 15% to about 45%, about 15% to about 50%, about 20% to about 25%, about 20% to about 30%, about 20% to about 35%, about 20% to about 40%, about 20% to about 45%, about 20% to about 50%, about 25% to about 30%, about 25% to about 35%, about 25% to about 40%, about 25% to about 45%, about 25% to about 50%, about 30% to about 35%, about 30% to about 40%, about 30% to about 45%, about 30% to about 50%, about 35% to about 40%, about 35% to about 45%, about 35% to about 50%, about 40% to about 45%, about 40% to about 50%, or about 45% to about 50%. In some embodiments, the methods yield a hematocrit percentage of red blood cells (RBCs) in a sample consisting of target cells and red blood cells (RBCs) of about 0.5%, about 1%, about 5%, about 10%, about 15%, about 20%, about 25%, about 30%, about 35%, about 40%, about 45%, or about 50%. In some embodiments, the methods yield a hematocrit percentage of red blood cells (RBCs) in a sample consisting of target cells and red blood cells (RBCs) of at least about 0.5%, about 1%, about 5%, about 10%, about 15%, about 20%, about 25%, about 30%, about 35%, about 40%, or about 45%. In some embodiments, maintaining an effective hematocrit can be achieved by adding red blood cells to a composition of target cells to achieved or produce the effective ratio.

Methods for maintaining an effective ratio of red blood cells (RBCs) to total cells (target and non-target cells) of the composition are useful for generating a cell population comprising target cells and having improved quantitative and qualitative properties for producing a cell therapy. Accordingly, methods yielding an effective ratio of red blood cells (RBCs) to target cells are useful for generating a collected target cell product (e.g. a cell population) having improved quantitative and qualitative properties for producing a cell therapy. In some embodiments, the method yields a ratio of red blood cells (RBCs) to total cells of about 1:1 to about 1,000:1. In some embodiments, the methods yield a ratio of red blood cells (RBCs) to total cells of about 1:1 to about 10:1, about 1:1 to about 50:1, about 1:1 to about 100:1, about 1:1 to about 200:1, about 1:1 to about 300:1, about 1:1 to about 400:1, about 1:1 to about 500:1, about 1:1 to about 600:1, about 1:1 to about 700:1, about 1:1 to about 800:1, about 1:1 to about 1,000:1, about 10:1 to about 50:1, about 10:1 to about 100:1, about 10:1 to about 200:1, about 10:1 to about 300:1, about 10:1 to about 400:1, about 10:1 to about 500:1, about 10:1 to about 600:1, about 10:1 to about 700:1, about 10:1 to about 800:1, about 10:1 to about 1,000:1, about 50:1 to about 100:1, about 50:1 to about 200:1, about 50:1 to about 300:1, about 50:1 to about 400:1, about 50:1 to about 500:1, about 50:1 to about 600:1, about 50:1 to about 700:1, about 50:1 to about 800:1, about 50:1 to about 1,000:1, about 100:1 to about 200:1, about 100:1 to about 300:1, about 100:1 to about 400:1, about 100:1 to about 500:1, about 100:1 to about 600:1, about 100:1 to about 700:1, about 100:1 to about 800:1, about 100:1 to about 1,000:1, about 200:1 to about 300:1, about 200:1 to about 400:1, about 200:1 to about 500:1, about 200:1 to about 600:1, about 200:1 to about 700:1, about 200:1 to about 800:1, about 200:1 to about 1,000:1, about 300:1 to about 400:1, about 300:1 to about 500:1, about 300:1 to about 600:1, about 300:1 to about 700:1, about 300:1 to about 800:1, about 300:1 to about 1,000:1, about 400:1 to about 500:1, about 400:1 to about 600:1, about 400:1 to about 700:1, about 400:1 to about 800:1, about 400:1 to about 1,000:1, about 500:1 to about 600:1, about 500:1 to about 700:1, about 500:1 to about 800:1, about 500:1 to about 1,000:1, about 600:1 to about 700:1, about 600:1 to about 800:1, about 600:1 to about 1,000:1, about 700:1 to about 800:1, about 700:1 to about 1,000:1, or about 800:1 to about 1,000:1. In some embodiments, the ratio of red blood cells (RBCs) to total cells of about 1:1, about 10:1, about 50:1, about 100:1, about 200:1, about 300:1, about 400:1, about 500:1, about 600:1, about 700:1, about 800:1, or about 1,000:1. In some embodiments, the ratio of red blood cells (RBCs) to total cells is at least about 1:1, about 10:1, about 50:1, about 100:1, about 200:1, about 300:1, about 400:1, about 500:1, about 600:1, about 700:1, or about 800:1. In some embodiments, maintaining an effective ratio of red blood cells (RBCs) to total cells can be achieved by adding red blood cells to a composition of target cells to achieved or produce the effective ratio.

Methods yielding platelet-reduced compositions comprising an effective ratio of platelets to target cells are useful in combination with the effective hematocrit percentage of red blood cells (RBCs) in a cell population, for generating a collected target cell product (e.g. a cell population) can provide improved quantitative and qualitative properties for producing a cell therapy. In some embodiments, in addition to the disclosed effective hematocrit percentage of red blood cells (RBCs) in a sample, the ratio of platelets to target cells less than about 500:1. In some embodiments, in addition to the disclosed effective hematocrit percentage of red blood cells (RBCs) in a sample, the ratio of platelets to target cells about 0.001:1 to about 0.01:1, about 0.001:1 to about 0.1:1, about 0.001:1 to about 1:1, about 0.001:1 to about 10:1, about 0.001:1 to about 25:1, about 0.001:1 to about 50:1, about 0.001:1 to about 100:1, about 0.001:1 to about 200:1, about 0.001:1 to about 300:1, about 0.001:1 to about 400:1, about 0.001:1 to about 500:1, about 0.01:1 to about 0.1:1, about 0.01:1 to about 1:1, about 0.01:1 to about 10:1, about 0.01:1 to about 25:1, about 0.01:1 to about 50:1, about 0.01:1 to about 100:1, about 0.01:1 to about 200:1, about 0.01:1 to about 300:1, about 0.01:1 to about 400:1, about 0.01:1 to about 500:1, about 0.1:1 to about 1:1, about 0.1:1 to about 10:1, about 0.1:1 to about 25:1, about 0.1:1 to about 50:1, about 0.1:1 to about 100:1, about 0.1:1 to about 200:1, about 0.1:1 to about 300:1, about 0.1:1 to about 400:1, about 0.1:1 to about 500:1, about 1:1 to about 10:1, about 1:1 to about 25:1, about 1:1 to about 50:1, about 1:1 to about 100:1, about 1:1 to about 200:1, about 1:1 to about 300:1, about 1:1 to about 400:1, about 1:1 to about 500:1, about 10:1 to about 25:1, about 10:1 to about 50:1, about 10:1 to about 100:1, about 10:1 to about 200:1, about 10:1 to about 300:1, about 10:1 to about 400:1, about 10:1 to about 500:1, about 25:1 to about 50:1, about 25:1 to about 100:1, about 25:1 to about 200:1, about 25:1 to about 300:1, about 25:1 to about 400:1, about 25:1 to about 500:1, about 50:1 to about 100:1, about 50:1 to about 200:1, about 50:1 to about 300:1, about 50:1 to about 400:1, about 50:1 to about 500:1, about 100:1 to about 200:1, about 100:1 to about 300:1, about 100:1 to about 400:1, about 100:1 to about 500:1, about 200:1 to about 300:1, about 200:1 to about 400:1, about 200:1 to about 500:1, about 300:1 to about 400:1, about 300:1 to about 500:1, or about 400:1 to about 500:1. In some embodiments, in addition to the disclosed effective hematocrit percentage of red blood cells (RBCs) in a sample, the ratio of platelets to target cells about 0.001:1, about 0.01:1, about 0.1:1, about 1:1, about 10:1, about 25:1, about 50:1, about 100:1, about 200:1, about 300:1, about 400:1, or about 500:1. In some embodiments, in addition to the disclosed effective hematocrit percentage of red blood cells (RBCs) in a sample, the ratio of platelets to target cells at most about 0.01:1, about 0.1:1, about 1:1, about 10:1, about 25:1, about 50:1, about 100:1, about 200:1, about 300:1, about 400:1, or about 500:1.

In many instances, separation methodologies must be performed under conditions that ensure non-contamination of the sample or maintain sterility. For example, many current clinical cell separation systems need to be operated in clean rooms of high quality in order to maintain sterility of samples. Alternatively, or in addition, samples can be processed in a “closed” system where the samples are not exposed to an outside environment. Often ensuring non-contamination is cumbersome, expensive and requires separate facilities and personnel, as well as complex procedures requiring extensive efforts to maintain reproducibility and sterility. Additionally, numerous processing and handling steps (e.g., washing, volume reduction, etc.) must be performed separate from the separation systems with ‘subsequent introduction of the processed samples as well as attachment of fluids and reagents to the systems, further complicating sterility compliance. As such, presented are improved methods and systems to ensure non-contamination of samples and/or reducing the complexity and expense of sample processing.

Following isolation, purification, or collection of compositions comprising red blood cells (RBCs) and target (and optionally, non-target cells), the target cells may be expanded in vitro, genetically engineered, or otherwise modified in order to confer a therapeutic effect to an individual subsequently treated with the cell. When the target cells are T cells, the target cells can be expanded in vitro using methods known in the art, such as stimulation with anti-CD3/anti-CD28 and/or cocktails of cytokines that may comprise inter alia IL-2, IL-15, IL-12, or IL-7.

Target cells may be engineered by rendering them transgenic for a nucleic acid that encodes a therapeutic protein. Such nucleic acids may comprise a therapeutic protein under the control of an inducible, tissue specific, or constitutive promoter. Such nucleic acids may also comprise additional features such as enhancer sequences or polyadenylation signals. In certain embodiments, the therapeutic protein may be a chimeric antigen receptor, recombinant T cell receptor, a cytokine, a chemokine, or an enzyme. Target cells may also be modified by siRNAs, shRNAs, or miRNAs. The cells may be rendered transgenic using various techniques including viral transduction, electroporation, or chemically mediated transfection reagents.

For genetic modification of target cells, a nucleic acid vector may be used for the delivery of a foreign nucleic acid into human cells. Exemplary methods to accomplish gene incorporation with vectors, include viral systems and non-viral systems. The major vectors for gene therapy in basic research and clinical study are viruses, due to the high transfer efficiency, the relatively short time needed to reach the clinically necessary numbers of cultured cells and the availability of different viruses with different expression characteristics. Viral systems can accommodate therapeutically useful genes, and constructs, and can provide the viral structural enzymes and proteins to allow for the generation of vector-containing infectious viral particles. The virus vectors include retroviruses (including lentivirus), adenovirus and adeno-associated virus. Among them, the most popular tools for gene delivery are genetically engineered retroviruses and/or lentiviruses.

Target cells may be genetically modified using a non-viral method. Non-viral gene therapy has maintained its position as an approach for treating cancer because of its higher efficiency, target specificity, non-infectiousness, unlimited carrier capacity, controlled chemical constitution and generous production. Non-viral vectors include naked DNA, liposomes, polymerizers and molecular conjugates. Minicircle DNA vectors free of plasmid bacterial DNA sequences may also be used as a non-viral vector for genetic modification. Non-viral methods also include electroporation.

The methods of expanding or genetically modifying target cells from reduced platelet blood related samples can be used subsequent to target cell expansion and/or genetic modification steps. In some embodiments, the red blood cells and target cells are present in the compositions subjected to expansion and genetic modification. In some embodiments, the red blood cells and target are present in the compositions subjected expansion of target cells. In some embodiments, the red blood cells and target are present in the compositions subjected to genetic modification. Alternatively, target cells may be isolated from a composition comprising red blood cells before genetic modification.

The target cells may be modified by a nucleic acid encoding a chimeric antigen receptor.

The target cells may be modified by a nucleic acid encoding a chimeric antigen receptor that binds to CD19.

The target cells may be modified by a nucleic acid encoding a chimeric antigen receptor that binds to CD20.

The target cells may be T cells modified by a nucleic acid encoding a chimeric antigen receptor.

The target cells may be T cells modified by a nucleic acid encoding a chimeric antigen receptor that binds to CD19.

The target cells may be T cells modified by a nucleic acid encoding a chimeric antigen receptor that binds to CD20.

A single method or combination of one or more methods can be employed to achieve the improved target cell compositions disclosed herein. Such methods include, but are not limited to, density gradient separation, deterministic lateral displacement, dielectrophoretic separation, acoustophoretic separation, and affinity separation. In some embodiments, the compositions disclosed herein are generated using density gradient separation, deterministic lateral displacement, dielectrophoretic separation, acoustophoretic separation, affinity separation, or any combination thereof In some embodiments, maintaining or adjusting the red blood cells in a cell population comprising target cells in achieved by density gradient separation, deterministic lateral displacement, dielectrophoretic separation, acoustophoretic separation, affinity separation, or any combination thereof. In some embodiments, processing methods disclosed herein comprise one or more steps, wherein the one or more steps can comprise density gradient separation, deterministic lateral displacement, dielectrophoretic separation, acoustophoretic separation, affinity separation, or any combination thereof. For example, the methods for ordered processing can first comprise a density separation step followed by a process that employs the separation of cells based on an array of microstructures based and pore sizes.

### Density Gradient Separation

Methods comprising centrifugal apheresis separates the plasma from cellular components based on density can be useful for obtaining one or more target cells from a blood related sample. Density gradient separation apheresis devices are designed to separate plasma or blood components from whole blood, for the purposes of depletion or exchange of these components or plasma. Density gradient separation comprises drawing whole blood from a patient and separating the blood into its components, utilizing centrifugal force as the basis of operation. Centrifugal flow devices most commonly deliver continuous flow from the patient to the centrifuge. An anticoagulant, usually citrate, is added before centrifugation, which is then followed by return of the rest of the blood components with the appropriate replacement fluid (typically albumin or plasma) so that a continuous flow extracorporeal circuit is formed.

Accordingly, density gradient separation can be used for generating a reduced platelet blood related sample. In certain embodiments, the reduced platelet blood related sample comprises a ratio of platelets to target cells of less than about 500:1. In certain embodiments, the reduced platelet blood related sample comprises a ratio of platelets to target cells of less than about 100:1. In certain embodiments, the reduced platelet blood related sample comprises a ratio of platelets to target cells of less than about 10:1. In certain embodiments, the reduced platelet blood related sample comprises a ratio of platelets to target cells of less than about 5:1. In certain embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1.

Density gradient separation can be used for obtaining one or more target cells from a blood related sample, wherein said blood related sample comprises the target cells, platelet cells, and red blood cells, said method comprising the steps of (a) reducing a number of the platelet cells from the blood related sample, to produce a reduced platelet blood related sample; and (b) adjusting a concentration of the red blood cells of the reduced platelet blood related sample to produce an adjusted red blood cell, reduced platelet blood related sample. In some embodiments, the adjusted red blood cell, reduced platelet blood related sample comprises from about 1×10³ red blood cells per microliter to about 1×10⁷ per microliter (uL). In some embodiments, the reduced platelet blood related sample comprises less than 10%, 5%, 2%, or 1% platelets compared to the blood related sample. In some embodiments, adjusting the concentration of the red blood cells comprises removing the red blood cells from the reduced platelet blood related sample or adding a diluent to the reduced platelet blood related sample. The adjusted red blood cell and reduced platelet blood related sample can comprise at least about 1×10⁴, 5×10⁴, 1×10⁵, 5×10⁵, 1×10⁶, 5×10⁶ red blood cells per microliter (uL). Additionally, red blood cells can be added to a collected cell target product from density gradient separation. In some embodiments, density gradient separation is used for the isolation of peripheral blood mononuclear cells (PBMCs). In certain embodiments, the isolation of peripheral blood mononuclear cells (PBMCs) is used for the isolation of T cells for the generation of chimeric antigen receptor T cells (CAR-T cells).

### Array-Based Separation

Methods utilizing arrays comprising microstructures (e.g. microposts or columns) that construct pores that separate cells based on critical sizes. For example, such methods generally utilize size exclusion to prevent or restrict entrance or passage by physical blockage. Embodiments of size exclusion comprise the use of small pores to prevent large non-deformable particles from entering the pores. The pore size can be engineered to allow for the separation of particles of different sizes (critical sizes). Such methods can also utilize laminar flow, tangential flow, or cross flow dynamics to facilitate sample processing. Accordingly, density gradient separation can be used for generating the target cell compositions disclosed herein.

For example, methods comprising Deterministic Lateral Displacement (DLD) for separating different cell types can be useful for obtaining one or more target cells from a blood related sample. DLD is a process in which particles are deflected on a path through an array, deterministically, based on their size in relation to some of the array parameters. DLD can also be used to concentrate cells and for buffer exchange. Processes are generally described herein in terms of continuous flow (DC conditions; i.e., bulk fluid flow in only a single direction). However, DLD can also work under oscillatory flow (AC conditions; i.e., bulk fluid flow alternating between two directions). DLD generally functions to separate cells or components thereof base on the critical size or predetermined size of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however, when the profile is asymmetrical, the critical sizes of the two sides of the gap can differ.

Basic principles of size based microfluidic separations and the design of obstacle arrays for separating cells have been provided elsewhere (see, US 2014/0342375; US 2016/0139012; U.S. Pat. Nos. 7,318,902 and 7,150,812, which are hereby incorporated herein in their entirety) and are also summarized in the sections below.

Procedures for making and using microfluidic devices that are capable of separating cells on the basis of size have also been fully described in the art. Such devices include those described in U.S. Pat. Nos. 75,837,115; 7,150,812; 6,685,841; 7,318,902; 7,472,794; and 7,735,652; all of which are hereby incorporated by reference in their entirety. Other references that provide guidance that may be helpful in the making and use of devices for the present invention include: U.S. Pat. Nos. 75,427,663; 7,276,170; 6,913,697; 7,988,840; 8,021,614; 8,282,799; 8,304,230; 8,579,117; US 2006/0134599; US 2007/0160503; US 20050282293; US 2006/0121624; US 2005/0266433; US 2007/0026381; US 2007/0026414; US 2007/0026417; US 2007/0026415; US 2007/0026413; US 2007/0099207; US 2007/0196820; US 2007/0059680; US 2007/0059718; US 2007/005916; US 2007/0059774; US 2007/0059781; US 2007/0059719; US 2006/0223178; US 2008/0124721; US 2008/0090239; US 2008/0113358; and WO2012094642 all of which are also incorporated by reference herein in their entirety.

Separation of cells in a sample can be performed by positive or negative selection of cell types using DLD and be collected in an output tube. Accordingly, DLD can be used for generating a reduced platelet blood related sample. In certain embodiments, the reduced platelet blood related sample comprises a ratio of platelets to target cells of less than about 500:1. In certain embodiments, the reduced platelet blood related sample comprises a ratio of platelets to target cells of less than about 100:1. In certain embodiments, the reduced platelet blood related sample comprises a ratio of platelets to target cells of less than about 10:1. In certain embodiments, the reduced platelet blood related sample comprises a ratio of platelets to target cells of less than about 5:1. In certain embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1.

DLD can also be used for obtaining one or more target cells from a blood related sample, wherein said blood related sample comprises the target cells, platelet cells, and red blood cells, said method comprising the steps of (a) reducing a number of the platelet cells from the blood related sample, to produce a reduced platelet blood related sample; and (b) adjusting a concentration of the red blood cells of the reduced platelet blood related sample to produce an adjusted red blood cell, reduced platelet blood related sample. In some embodiments, the adjusted red blood cell, reduced platelet blood related sample comprises from about 1×10³ red blood cells per microliter to about 1×10⁷ per microliter (uL). In some embodiments, the reduced platelet blood related sample comprises less than 10%, 5%, 2%, or 1% platelets compared to the blood related sample. In some embodiments, adjusting the concentration of the red blood cells comprises removing the red blood cells from the reduced platelet blood related sample or adding a diluent to the reduced platelet blood related sample. The adjusted red blood cell and reduced platelet blood related sample can comprise at least about 1×10⁴, 5×10⁴, 1×10⁵, 5×10⁵, 1×10⁶, 5×10⁶ red blood cells per microliter (uL). Additionally, red blood cells can be added to a collected cell target product from DLD. In some embodiments, the sample is a blood sample. In some embodiments, DLD is used for the isolation of peripheral blood mononuclear cells (PBMCs). In certain embodiments, the isolation of peripheral blood mononuclear cells (PBMCs) is used for the isolation of T cells for the generation of chimeric antigen receptor T cells (CAR-T cells).

### Dielectrophoresis

Methods comprising dielectrophoresis (DEP) for separating different cell types can be useful for obtaining one or more target cells from a blood related sample. Dielectrophoresis (DEP) is a phenomenon in which particles, or cells, exposed to the gradient of an electric field are polarized depending on the characteristics of the cells and the medium that surrounds them. See U.S. Pat. No. 10,078,066; See also Douglas T A et al. “Separation of Macrophages and Fibroblasts Using Contactless Dielectrophoresis and a Novel ImageJ Macro.” Bioelectricity. 2019; 1(1):49-55. doi:10.1089/bioe.2018.0004. Such polarization induces movement of the cells along the gradient of the electric field. Accordingly, dielectrophoresis (DEP) can be used to trap cells or divert them from normal streamlines. For example, dielectrophoresis (DEP) can be used to positively or negatively select target cell from a population of cells. Contactless dielectrophoresis (DEP), which employs a polydimethylsiloxane (PDMS) microfluidic device containing a cell flow chamber can be used to facilitate dielectrophoresis (DEP) isolation of cell types. A polydimethylsiloxane (PDMS) microfluidic device generally comprises a chamber containing an array of 20 mircometer (um) posts where cells trap based on the gradient of an applied electric field. The device also generally comprises contactless fluidic electrodes that are filled with conductive fluid and separated from the main channel by a thin polydimethylsiloxane (PDMS) membrane. Applying voltage using contactless electrodes filled with a concentrated buffer (e.g. 10× concentrated phosphate-buffered saline (PBS)) eliminates problems with cell mortality as is seen in traditional dielectrophoresis by preventing electrolysis and bubble formation in the microfluidic device, as well as avoiding contact between regions of high electric field and cells.

In addition to improving cellular viability, utilizing small post structures allows better control of cell selectivity by preventing pearl chaining and cell-cell interactions. Cells with different bioelectrical phenotypes are trapped in the main channel at different applied electric field frequencies. By modulating the applied frequency, the device can selectively trap some cells while allowing others to pass through the device. This selectivity allows separation of highly similar cell types in a label-free manner while maintaining high cellular viability such that they can be cultured or further characterized downstream. This method provides more selective and higher viability separation of cells, which allows more closely related and physically similar cells to be separated, while allowing less similar cells to be separated at a much higher efficiency.

Batch separation can be performed by trapping some of the cells while allowing other cells to flow through and be collected in an output tube. After turning off the voltage, trapped cells can be released from their posts and can be collected in another output tube. Accordingly, dielectrophoresis can be used for generating a reduced platelet blood related sample. In certain embodiments, the reduced platelet blood related sample comprises a ratio of platelets to target cells of less than about 500:1. In certain embodiments, the reduced platelet blood related sample comprises a ratio of platelets to target cells of less than about 100:1. In certain embodiments, the reduced platelet blood related sample comprises a ratio of platelets to target cells of less than about 10:1. In certain embodiments, the reduced platelet blood related sample comprises a ratio of platelets to target cells of less than about 5:1. In certain embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1. Dielectrophoresis can also be used for obtaining one or more target cells from a blood related sample, wherein said blood related sample comprises the target cells, platelet cells, and red blood cells, said method comprising the steps of (a) reducing a number of the platelet cells from the blood related sample, to produce a reduced platelet blood related sample; and (b) adjusting a concentration of the red blood cells of the reduced platelet blood related sample to produce an adjusted red blood cell, reduced platelet blood related sample. This can be achieved by modulating or tuning the applied electrical field frequencies to achieve (a) and (b). In some embodiments, the adjusted red blood cell, reduced platelet blood related sample comprises from about 1×10³ red blood cells per microliter to about 1×10⁷ per microliter (uL). In some embodiments, the reduced platelet blood related sample comprises less than 10%, 5%, 2%, or 1% platelets compared to the blood related sample. In some embodiments, adjusting the concentration of the red blood cells comprises removing the red blood cells from the reduced platelet blood related sample or adding a diluent to the reduced platelet blood related sample. The adjusted red blood cell and reduced platelet blood related sample can comprise at least about 1×10⁴, 5×10⁴, 1×10⁵, 5×10⁵, 1×10⁶, 5×10⁶ red blood cells per microliter (uL). Additionally, red blood cells can be added to a collected cell target product from dielectrophoresis. In some embodiments, dielectrophoresis is used for the isolation of peripheral blood mononuclear cells (PBMCs). In certain embodiments, the isolation of peripheral blood mononuclear cells (PBMCs) is used for the isolation of T cells for the generation of chimeric antigen receptor T cells (CAR-T cells).

### Acoustophoretic Isolation

Methods comprising acoustophoresis for separating different cell types can be useful for obtaining one or more target cells from a blood related sample. Acoustophoresis is a phenomenon in which cells, exposed to an acoustic pressure field, are separated based on the characteristics of the cells. See U.S. Pat. No. 10,640,760; See also Dutra, Brian et al. “A Novel Macroscale Acoustic Device for Blood Filtration.” Journal of medical devices vol. 12,1 (2018): 0110081-110087. doi:10.1115/1.4038498. The underlying principle of the acoustic separation is based on the nonuniform acoustic pressure field in the fluid established by an acoustic standing wave. The introduction of a particle in this acoustic pressure field leads to a scattering of the acoustic pressure. The acoustic pressure acting on the surface of the particle then consists of the sum of the incident acoustic standing wave and the scattered wave. The net time averaged force on the particle is determined by integrating the acoustic pressure on the surface of the particle (i.e. acoustic radiation force). In addition to the axial acoustic radiation force component, a three-dimensional acoustic wave also exerts lateral forces on the suspended particle, orthogonal to the axis. An axial component of the acoustic radiation force component directs particles to collect in planes at the pressure nodes or antinodes every half wavelength, determined by a positive or negative acoustic contrast factor, respectively. A lateral component of the acoustic radiation force component collects the cells within the planes to local clusters, where the cells grow in collective size until they reach critical mass and the gravity/buoyancy force causes the cells to sink or rise out of suspension, thus separating the cells.

Separation of cells in a sample can be performed by positive or negative selection of cell types using acoustophoresis and be collected in an output tube. Accordingly, acoustophoresis can be used for generating a reduced platelet blood related sample. In certain embodiments, the reduced platelet blood related sample comprises a ratio of platelets to target cells of less than about 500:1. In certain embodiments, the reduced platelet blood related sample comprises a ratio of platelets to target cells of less than about 100:1. In certain embodiments, the reduced platelet blood related sample comprises a ratio of platelets to target cells of less than about 10:1. In certain embodiments, the reduced platelet blood related sample comprises a ratio of platelets to target cells of less than about 5:1. In certain embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1. Acoustophoresis can also be used for obtaining one or more target cells from a blood related sample, wherein said blood related sample comprises the target cells, platelet cells, and red blood cells, said method comprising the steps of (a) reducing a number of the platelet cells from the blood related sample, to produce a reduced platelet blood related sample; and (b) adjusting a concentration of the red blood cells of the reduced platelet blood related sample to produce an adjusted red blood cell, reduced platelet blood related sample. This can be achieved by modulating or tuning the applied acoustic field frequencies to achieve (a) and (b). In some embodiments, the adjusted red blood cell, reduced platelet blood related sample comprises from about 1×10³ red blood cells per microliter to about 1×10⁷ per microliter (uL). In some embodiments, the reduced platelet blood related sample comprises less than 10%, 5%, 2%, or 1% platelets compared to the blood related sample. In some embodiments, adjusting the concentration of the red blood cells comprises removing the red blood cells from the reduced platelet blood related sample or adding a diluent to the reduced platelet blood related sample. The adjusted red blood cell and reduced platelet blood related sample can comprise at least about 1×10⁴, 5×10⁴, 1×10⁵, 5×10⁵, 1×10⁶, 5×10⁶ red blood cells per microliter (uL). Additionally, red blood cells can be added to a collected cell target product from acoustophoresis. In some embodiments, the sample is a blood sample. In some embodiments, acoustophoresis is used for the isolation of peripheral blood mononuclear cells (PBMCs). In certain embodiments, the isolation of peripheral blood mononuclear cells (PBMCs) is used for the isolation of T cells for the generation of chimeric antigen receptor T cells (CAR-T cells).

### Affinity Separation

Various techniques are known for separating components of a sample or biological material that make use of affinity-based separation techniques. Immunoaffinity methods may include selective labeling of certain components of a sample (e.g., antibody labeling) and separation of labeled and unlabeled components. To isolate cells from a biological sample, either pre-enriched or not, immunoaffinity capture utilizing an affinity molecule (e.g. an antibody, binding protein, aptamer, etc.) is used. Accordingly, immunoaffinity capture is used herein to refer to the use of affinity molecules (e.g. an antibody, binding protein, aptamer, etc.) to capture or isolate cells from a sample. Affinity molecules (e.g. an antibody, binding protein, aptamer, etc.) that bind specific cell marker proteins function as ligands to target cells, thereby providing a means to capture cells (either directly or indirectly) and permit their isolation from the sample. Examples of immunoaffinity capture techniques include, but are not limited to, immunoprecipitation, column affinity chromatography, magnetic-activated cell sorting, fluorescence-activated cell sorting, adhesion-based sorting and microfluidic-based sorting, either directly or using carriers. Affinity molecules (e.g. an antibody, binding protein, aptamer, etc.) in a homogeneous or a heterogenous cocktail may be utilized together, in a single solution, or may be utilized in two or more solutions that are used simultaneously or consecutively.

Magnetic separation methods typically include passing the sample through a separation column or incubation with a bead-based solution. Magnetic separation is a procedure for selectively retaining magnetic materials in a chamber or column disposed in a magnetic field. A target substance, including biological materials, may be magnetically labeled by attachment to a magnetic particle by means of a specific binding partner, which is conjugated to the particle. A suspension of the labeled target substance is then applied to the chamber. The target substance is retained in the chamber in the presence of a magnetic field. The retained target substance can then be eluted by changing the strength of, or by eliminating, the magnetic field. A matrix of material of suitable magnetic susceptibility may be placed in the chamber, such that when a magnetic field is applied to the chamber a high magnetic field gradient is locally induced close to the surface of the matrix. This permits the retention of weakly magnetized particles and the approach is referred to as high gradient magnetic separation (HGMS).

Separation of cells in a sample can be performed by positive or negative selection of cell types using affinity purification and be collected in an output tube. Accordingly, affinity separation can be used for generating a reduced platelet blood related sample. In certain embodiments, the reduced platelet blood related sample comprises a ratio of platelets to target cells of less than about 500:1. In certain embodiments, the reduced platelet blood related sample comprises a ratio of platelets to target cells of less than about 100:1. In certain embodiments, the reduced platelet blood related sample comprises a ratio of platelets to target cells of less than about 10:1. In certain embodiments, the reduced platelet blood related sample comprises a ratio of platelets to target cells of less than about 5:1. In certain embodiments, the red blood cells are maintained at a ratio of red blood cells to target cells of greater than about 100:1. In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1. Affinity purification can also be used for obtaining one or more target cells from a blood related sample, wherein said blood related sample comprises the target cells, platelet cells, and red blood cells, said method comprising the steps of (a) reducing a number of the platelet cells from the blood related sample, to produce a reduced platelet blood related sample; and (b) adjusting a concentration of the red blood cells of the reduced platelet blood related sample to produce an adjusted red blood cell, reduced platelet blood related sample. This can be achieved through the introduction of affinity molecules (e.g. an antibody, binding protein, aptamer, etc.) capable binding a desired target and positively or negatively selecting cells to achieve (a) and (b). Affinity molecules (e.g. an antibody, binding protein, aptamer, etc.) that bind biomarkers on the surface of platelets are thus useful. Known platelet surface biomarkers include, but are not limited to, CD36, CD41 (GP IIb/IIIa), CD42a (GPIX), CD42b (GPIb), and CD61 (avb3, vitronectin receptor). Known platelet activation biomarkers appear on the platelet surface during activation and can be targeted. Platelet activation biomarkers include, but are not limited to, PAC-1 (activated IIb/IIIa), CD62P (P-selectin), CD31 (PECAM) and CD63. Red blood cell surface biomarkers can be useful for the targeting of affinity molecules (e.g. an antibody, binding protein, aptamer, etc.). Known red blood cell biomarkers include, but are not limited to, surface antigen A, surface antigen B, Rh factor, and CD235a.

In some embodiments, the adjusted red blood cell, reduced platelet blood related sample comprises from about 1×10³ red blood cells per microliter to about 1×10⁷ per microliter (uL). In some embodiments, the reduced platelet blood related sample comprises less than 10%, 5%, 2%, or 1% platelets compared to the blood related sample. In some embodiments, adjusting the concentration of the red blood cells comprises removing the red blood cells from the reduced platelet blood related sample or adding a diluent to the reduced platelet blood related sample. The adjusted red blood cell and reduced platelet blood related sample can comprise at least about 1×10⁴, 5×10⁴, 1×10⁵, 5×10⁵, 1×10⁶, 5×10⁶ red blood cells per microliter (uL). In some embodiments, red blood cells are added to collected target cell products from affinity purification. In some embodiments, the sample is a blood sample. In some embodiments, affinity separation is used for the isolation of peripheral blood mononuclear cells (PBMCs). In certain embodiments, the isolation of peripheral blood mononuclear cells (PBMCs) is used for the isolation of T cells for the generation of chimeric antigen receptor T cells (CAR-T cells).

### Target Cells

Target cells comprise a type of cell, cell population, or composition of cells which are the desired cells to be collected, isolated, or separated by the present invention. Generally, as disclosed herein, target cells can be any cell intended for immediate or downstream therapeutic use. The target cells disclosed herein are eukaryotic cells and generally consist of immune cells. Immune cells comprise cells originating from myeloid or lymphocyte lineages. In some embodiments, the therapeutic cell is a lymphocyte. The lymphocyte comprises natural killer cells, T cells, and B cells. In certain embodiments, the target cell is a natural killer cell (e.g., CD56+ or CD16+). In some embodiments, the target cell is a T cell. In some embodiments, the target cell is a CD4+ T cell. In some embodiments, the target cell is a CD8+ T cell. In some embodiments, the target cell is a central memory T cell (e.g., CCR7+ CD45RA−CD45RO+CD62L+CD27+). In some embodiments, the T cell is CCR7+. In some embodiments, the T cell is CD62L+. In some embodiments, the T cell is CD45RO+. Such positivity can be determined for example by flow cytometry compared to an isotype control or a cell population known to be negative for the specific marker. In some embodiments, the target cell is a myeloid cell. The myeloid cell lineage comprises neutrophils, eosinophil, basophils, monocytes, dendritic cells, and macrophages. In some embodiments, the therapeutic cell is an eosinophil, a basophil, a dendritic cell, a monocyte, a macrophage, a microglial cell, a Kupffer cell, or an alveolar macrophage.

The therapeutic cells described herein can be endogenous cells that have been isolated and enriched. In some embodiments, the therapeutic cells are derived from a subject. In some embodiments, the therapeutic cells are allogenic. Additionally, therapeutic cells can be derived from endogenous cells comprising pluripotent stem cells, hematopoietic stem cells, placental or fetal cells, from an adult human. The therapeutic cells can also be obtained from an established cell line or culture. In some embodiments, the therapeutic cells comprise cells derived from a cell line or established culture, wherein the cell line or established culture is derived from endogenous cells comprising pluripotent stem cells, hematopoietic stem cells, placental or fetal cells, from an adult human.

The therapeutic cells can also comprise engineered or modified cells. The cell types described herein can be modified to provide enhanced affinity, avidity, and/or specificity for a target. For example, modified natural killer cells can comprise a vector for encoding an Fc receptor molecule. Accordingly, a modified therapeutic cell comprises a vector that encodes for transgenes or a genetic modification, including a nucleic acid sequence that encodes for an Fc receptor. Therapeutic cells can also or additionally be modified to provide enhance effector functions (e.g. capacity to kill or eliminate a target cell).

Accordingly, the target cells can comprise stem cells or immune cells. In some embodiments, the cell is selected from the group consisting of a T cell, a Natural Killer (NK) cell, a human embryonic stem cell, and a pluripotent stem cell from which lymphoid cells may be differentiated. In certain embodiments, the cell is a T cell. In certain embodiments, the T cell is selected from the group consisting of a cytotoxic T lymphocyte (CTL), a regulatory T cell, and a Natural Killer T (NKT) cell. In certain embodiments, the immunoresponsive cell is a myeloid cell such as macrophage.

For example, some therapeutic applications such as CAR cell therapy or adoptive T cell therapies the sample may be an autologous sample for an individual to be treated. Also contemplated are blood related samples from an individual ultimately to be treated with a stem cell transplant or therapeutic cell. Also contemplated are samples from a family member, monozygotic twin, or otherwise HLA matched donor, providing cells for the therapeutic treatment of another individual (e.g., heterologous samples). A sample for processing may have been subjected to one or more steps to prepare the sample for processing or to facilitate collection of the sample, including the addition of anti-coagulants or the depletion of one or more non-target cells. Suitable anticoagulants include citric acid, sodium citrate, dextrose, heparin, and chelating agents such as EDTA or EGTA. In certain embodiments, the sample may be treated with anti-coagulant citrate dextrose solution (ACD-A, citric acid monohydrate, dextrose monohydrate, and trisodium citrate dihydrate). Individuals from which the sample is collected may be administered a blood thinner, anti-coagulant, or anti-inflammatory drug before collection.

Target cells can be isolated from the reduced platelet blood related sample using any of the methods described herein to obtain a pure or partially pure population of target cells comprising at least about 50%, about 60%, about 70%, about 80% about 90%, about 95%, about 97%, about 98%, or about 99% purity. In certain embodiments, the methods described herein produce cell populations that are at least about 50%, about 60%, about 70%, about 80% about 90%, about 95%, about 97%, about 98%, or about 99% T lymphocytes (CD3+). In certain embodiments, the methods described herein produce cell populations that are at least about 50%, about 60%, about 70%, about 80% about 90%, about 95%, about 97%, about 98%, or about 99% central memory T lymphocytes (CD62L+or CCR7+).

The methods described herein can produce populations of target cells that exceed about 1×10⁶, about 2×10⁶, about 5×10⁶, about 1×10⁶, about 1×10⁷, about 2×10⁷, about 5×10⁷, about 1×10⁸, about 2×10⁸, about 5×10⁸, about 1×10⁹, about 2×10⁹, about 1×10¹⁰, about 2×10¹⁰, or about 5×10¹⁰ or more. The methods described herein can produce populations of T lymphocytes cells that exceed about 1×10⁶, about 2×10⁶, about 5×10⁶, about 1×10⁶, about 1×10⁷, about 2×10⁷, about 5×10⁷, about 1×10⁸, about 2×10⁸, about 5×10⁸, about 1×10⁹, about 2×10⁹, about 1×10¹⁰, about 2×10¹⁰, or about 5×10¹⁰ or more. The methods described herein can produce populations of central memory T lymphocytes cells that exceed about 1×10⁶, about 2×10⁶, about 5×10⁶, about 1×10⁶, about 1×10⁷, about 2×10⁷, about 5×10⁷, about 1×10⁸, about 2×10⁸, about 5×10⁸, about 1×10⁹, about 2×10⁹, about 1×10¹⁰, about 2×10¹⁰, or about 5×10¹⁰ or more.

### Non-Target Cells

Blood related samples comprise many non-target cell types that lack therapeutic activity or interfere with the therapeutic activity of a particular cell type. Non-target cell-types can be depleted or removed before reduction of platelets from a blood related sample, during adjustment/maintenance of a certain number of red blood cells, or after adjustment/maintenance of a certain number of red blood cells

In certain embodiments, non-target cells are removed or depleted from the blood-related sample before reducing a number of the platelet cells from the blood-related sample. In certain embodiments, non-target cells are removed or depleted from the reduced platelet blood related sample. In certain embodiments, non-target cells are removed or depleted from the adjusted red blood cell, reduced platelet blood related sample. In certain embodiments, where target cells are purified or isolated from an adjusted red blood cell, reduced platelet blood related sample non-target cells are not removed.

Non-target cell types can be removed by any suitable method. In certain embodiments, non-target cells are removed by an affinity-based method such as cell sorting based on a cell-surface marker associated with the non-target cell. For example, magnetic beads coupled to antibodies specific for cell-surface markers of non-target cells may be used in conjunction with a magnetic field to remove the non-target cells. Other methods for removing non-target cells including flow cytometry-based methods, deterministic lateral displacement (DLD) methods, acoustophoretic methods, dielectrophoretic methods, or methods based on size, density, or granularity may be used.

Non-target cell types according to this method will vary dependent upon the target cell isolated and the therapeutic purpose of the target cell isolated. For example, when the target cell is a memory T cell, any cell that is not a memory T cell (e.g. B cells or regulatory T cells (Tregs)) In instances wherein the target cell is a T lymphocyte, non-target cells may comprise any one or more of B lymphocytes, dendritic cells, monocytes, macrophages, granulocytes, basophils, eosinophils, neutrophils, mast cells, natural and killer cells. In instances wherein the target cell is a natural killer cell, non-target cells may comprise any one or more of B lymphocytes, dendritic cells, monocytes, macrophages, granulocytes, basophils, eosinophils, neutrophils, mast cells, and T cells. In instances wherein the target cell is a B lymphocyte, non-target cells may comprise any one or more of T lymphocytes, dendritic cells, monocytes, macrophages, granulocytes, basophils, eosinophils, neutrophils, mast cells, and natural and killer cells. In instances wherein the target cell is a dendritic cell, non-target cells may comprise any one or more of T lymphocytes, B lymphocytes, monocytes, macrophages, granulocytes, basophils, eosinophils, neutrophils, mast cells, and natural and killer cells. In instances wherein the target cell is a macrophage, non-target cells may comprise any one or more of T lymphocytes, dendritic cells, monocytes, B lymphocytes, granulocytes, basophils, eosinophils, neutrophils, mast cells, and natural and killer cells.

One or more immune suppressive non-target cells may be removed. Where target cells useful for their cytotoxic or inflammatory effect are desired (e.g. CAR cells, T cell receptor transgenic cells, dendritic cells or other APCs used to stimulate autologous or heterologous T cells) one or more suppressive cells that inhibit activation of T cells or other therapeutic cell types may be reduced or depleted. In certain embodiments, one or more of regulatory T cells, regulatory B cells, myeloid derived suppressor cells, or anti-inflammatory M2 macrophages may be removed. Regulatory T cells for example, can be identified based on expression of CD4 and CD25, GITR or FoxP3, and depleted by anti-CD25 or anti-GITR antibodies.

Subsets of cell types may be depleted in favor of target cells of another subset of the same cell type. The subset may be a T cell subset, a B cell subset, a macrophage subset, or a dendritic cell subset. For example, if CD8+ cytotoxic cells are desired CD4+ T cells or exhausted, naïve or suppressive CD8 T cells may be depleted. In certain embodiments, central memory T cells are the target cells and non-target cells comprise one or more of effector memory T cells, naïve T cells, exhausted T cells, or regulatory T cells are the non-target cell to be depleted. In certain embodiments, cells expressing any one or more of the exhausted or suppressive cell surface markers CD279/PD-1+, CD223/LAG-3+, TIGIT+, CD160+, CD152/CTL-4+, CD366/TIM-3 may be depleted.

Non-target cells can be removed by any of the methods disclosed herein. In some embodiments, the non-target cells are removed by negative selection methods. In some embodiments, the non-target cells are removed by positive selection methods. Non-target cells can also be removed from a sample prior to or after the generation of a target cell composition comprising target cells and red blood cells.

### Applications of Compositions and Methods for Improved Cell Therapy Manufacture

The compositions, methods, and systems disclosed herein are suited to facilitate the use of isolated or purified target cells in the application of cell-based therapies. As disclosed herein, the therapeutic eukaryotic cell can be derived from an induced pluripotent stem cell, a hematopoietic stem cell, or fetal cells. In some embodiments, the therapeutic eukaryotic cell is derived from an adult human. In some embodiments, the therapeutic eukaryotic cell comprises a natural killer (NK) cell, an NKT cell, a T cell, an eosinophil, a basophil, a dendritic cell, a monocyte, a macrophage, a microglial cell, a Kupffer cell, or an alveolar macrophage.

The cells for down-stream use in therapeutic applications can further be engineered with nucleic acids encoding one or more therapeutically useful proteins, including, but not limited to chimeric antigen receptors, recombinant T cell receptors, costimulatory/immunostimulatory molecules, or antigens (e.g., foreign or tumor associate antigens). In certain embodiments, cells are engineered using viral vectors, such as adenovirus, adeno-associated virus, or lentivirus vectors; CRISPR technology; or plasmid or linear nucleic acid stretches (e.g., rendered transgenic by electroporation or use of a lipid or cationic lipid-based transfection reagent).

The cells or cell types described herein can be modified to create, generate, or enhance binding of. For example, modified natural killer cells can comprise a vector for encoding an Fc receptor molecule. Genetic engineering of can be achieved by viral transduction or electroporation of transient nucleic acids (e.g. non-integrating expression plasmids or messenger ribonucleic acid). In some embodiments, the engineered cells are genetically modified wherein the genetic modification comprises alteration of the cell's genome. In some embodiments, the engineered cells are genetically modified wherein the genetic modification comprises introduction of a transient gene into the cell. Accordingly, an engineered cell comprises a vector that encodes for transgenes or a genetic modification, including a nucleic acid sequence that encodes for a chimeric antigen receptor. Engineered cells can also or additionally be modified to provide enhance effector functions (e.g. capacity to kill or eliminate a target cell). In some embodiments, the engineered cells additionally comprise stimulatory molecules. Such stimulatory molecules, for example, can enhance the killing and/or immune activation of an engineered cell or increase proliferation of the engineered cells. a suicide gene capable of killing the therapeutic cell upon administration of a drug or small molecule (e.g., a thymidine kinase gene, which can be antagonized by ganciclovir, valganciclovir, or acyclovir).

The compositions, methods, and systems disclosed provided collected target cell products (e.g. cell populations) that facilitate the generation of chimeric antigen receptor (CAR) T cells. Chimeric antigen receptor (CAR) T cell immunotherapy is a highly effective form of adoptive cell therapy, as demonstrated by the remission rates in patients with B cell acute lymphoblastic leukemia or large B cell lymphoma, which have supported FDA approvals.

Methods for making and using CAR T cells are known in the art. Procedures have been described in, for example, U.S. Pat. Nos. 9,629,877; 9,328,156; 8,906,682; US 2017/0224789; US 2017/0166866; US 2017/0137515; US 2016/0361360; US 2016/0081314; US 2015/0299317; and US 2015/0024482; each of which is incorporated by reference herein in its entirety.

For some therapeutic applications such as CAR cell therapy or adoptive T cell therapies the sample may be an autologous sample for an individual to be treated. Also contemplated are blood related samples from an individual ultimately to be treated with a stem cell transplant or therapeutic cell. Also contemplated are samples from a family member, monozygotic twin, or otherwise HLA matched donor, providing cells for the therapeutic treatment of another individual (e.g., heterologous samples). A sample for processing may have been subjected to one or more steps to prepare the sample for processing or to facilitate collection of the sample, including the addition of anti-coagulants or the depletion of one or more non-target cells. Suitable anticoagulants include citric acid, sodium citrate, dextrose, heparin, and chelating agents such as EDTA or EGTA. In certain embodiments, the sample may be treated with anti-coagulant citrate dextrose solution (ACD-A, citric acid monohydrate, dextrose monohydrate, and trisodium citrate dihydrate). Individuals from which the sample is collected may be administered a blood thinner, anti-coagulant, or anti-inflammatory drug before collection, plasma expanders including dextrans, other platelet activation inhibitors, or NETOSIS inhibitors.

The present invention includes methods of producing CAR T cells from samples of blood as well as from blood derived products such as apheresis or leukapheresis preparations. Procedures for genetically transforming T cells to express chimeric antigen receptors (CARs) on their surface are well established in the art. These receptors should generally bind antigens that are on the surface of a cell associated with a disease or abnormal condition. For example, the receptors may bind antigens that are unique to, or overexpressed on, the surface of cancer cells. Once produced, the CAR T cells may be expanded in number by growing the cells in vitro. Activators or other factors may be added during this process to promote growth, with IL-2 and IL-15 being among the agents that may be used.

Chimeric receptors will typically have a) an extracellular region with an antigen binding domain; b) a transmembrane region and c) an intracellular region. The cells may also be recombinantly engineered with sequences that provide the cells with a molecular switch that, when triggered, reduce CAR T cell number or activity. In a preferred embodiment, the antigen binding domain is a single chain variable fragment (scFv) from the antigen binding regions of both heavy and light chains of a monoclonal antibody. There is also preferably a hinge region of 2-20 amino acids connecting the extracellular region and the transmembrane region. The transmembrane region may have CD3 zeta, CD4, CD8, or CD28 protein sequences and the intracellular region should have a signaling domain, typically derived from CD3-zeta, CD137 or a CD28. Other signaling sequences may also be included that serve to regulate or stimulate activity.

CAR T cells made using the methods discussed herein may be used in treating patients for leukemia, e.g., acute lymphoblastic leukemia using procedures well established in the art of clinical medicine and, in these cases, the CAR may recognize CD19 or CD20 as a tumor antigen. The method may also be used for solid tumors, in which case antigens recognized may include CD22; RORI; mesothelin; CD33/IL3Ra; c-Met; PSMA; Glycolipid F77; EGFRvIII; GD-2; NY-ESO-1; MAGE A3; and combinations thereof. With respect to autoimmune diseases, CAR T cells may be used to treat rheumatoid arthritis, lupus, multiple sclerosis, ankylosing spondylitis, type 1 diabetes or vasculitis.

CAR technology can also be applied to other immune cells such as natural killer (NK) cells. NK cells are defined as CD56+ and CD3− cells and are subdivided into cytotoxic and immunoregulatory. They are of great clinical interest because they contribute to the graft-vs-leukemia/graft-vs-tumor effect but are not responsible for graft-vs-host disease. NK cells can be generated from various sources such as umbilical cord blood, bone marrow, human embryonic stem cells, and induced pluripotent stem cells. However, tumors can escape the cytotoxicity of NK cells when they are directed against NKG2D ligands MICA and MICB (major histocompatibility complex class I chain-related protein A/B).3Henceforth, preclinical research has been reported for CAR-modified primary human NK cells redirected against CD19, CD20, CD244, and HER2, as well as CAR-expressing NK-92 cells targeted to a wider range of cancer antigens.

NK cells can be directed with chimeric antigen receptor s to target surface molecules expressed by tumor cells. Accordingly, natural killer cells comprising an anti-ROR1 chimeric antigen receptor are useful for recognizing a cancer cell expressing ROR1 molecule and killing the ROR1 expressing cancer cell. In some embodiments, the therapeutic cell is a natural killer cell. In certain embodiments, the natural killer cell from an NK-92 cell line or derivative thereof In some embodiments, the NK cells express a biomarker associated with NK cells such as, CD56, CD2, CD7, CD11a, CD28, CD45, and CD54 surface markers. In some embodiments, the NK cell does not display the CD1, CD3, CD4, CD5, CD8, CD10, CD14, CD16, CD19, CD20, CD23, and CD34 markers. In some embodiments, the natural killer cell is from an NK-92, NK-YS, NK-YT, NK-YTS, NK-KHYG-1, NKL, NKG, SNK-6, or IMC cell line or a derivative thereof. Killer-cell immunoglobulin-like receptors (KIRs), are a family of type I transmembrane glycoproteins expressed on the plasma membrane of natural killer (NK) cells. KIRs regulate the killing function of NK cells by interacting with major histocompatibility (MHC) class I molecules, which are expressed on all nucleated cell types. Inhibitory KIR receptors down regulate the killing activity of NK cells. Therefore, in some embodiments, the NK cells do not express an inhibitory KIR receptor. In some embodiments, the NK cells do not express KIR2DL, KIR3DL, ILT2, ILT3, ILT4, ILT5, or LIR8. In some embodiments, the NK cells do not express an MHC molecule.

NK cells can further be engineered to promote activation and/or proliferation. Accordingly, NK cells can further comprise activating receptors that are expressed in addition an anti-ROR1 chimeric antigen receptor. For example, engineered NK cells can express or over express an IL-15 receptor molecule, wherein IL-15 can be administered to a patient in order to enhance the activation of the NK cell. In some embodiments, the NK cell further expresses an IL-15 receptor. In some embodiments, the NK cell further expresses an IL-2 receptor.

In some embodiments, the cell or engineered cell is a T cell. In some embodiments, the cell or engineered cell is a macrophage or monocyte.

In one embodiment described herein is a method of producing a chimeric antigen T cell comprising: a) providing a blood related sample comprising one or more T cells, platelet cells, red blood cells; and (b) reducing a number of the platelet cells in the blood related sample while maintaining a ratio of the red blood cells to the one or more T cells greater than about 50:1 to produce a reduced platelet blood related sample comprising the one or more target cells (c) optionally isolating the one or more T cells; (d) and transducing the one or more T cells with a nucleic acid encoding a chimeric antigen receptor. In certain embodiments, the chimeric antigen receptor comprises a binding domain which binds to: CD19; CD20; CD22; ROR1; mesothelin; CD33/IL3Ra; c-Met; PSMA; Glycolipid F77; EGFRvIII; GD-2; NY-ESO-1; MAGE A3, or combinations thereof. In certain embodiments, the chimeric antigen receptor comprises a binding domain which binds to CD19

In one embodiment described herein is a method of producing a chimeric antigen NK cell comprising: a) providing a blood related sample comprising one or more NK cells, platelet cells, red blood cells; and (b) reducing a number of the platelet cells in the blood related sample while maintaining a ratio of the red blood cells to the one or more NK cells greater than about 50:1 to produce a reduced platelet blood related sample comprising the one or more target cells (c) optionally isolating the one or more NK cells; (d) and transducing the one or more NK cells with a nucleic acid encoding a chimeric antigen receptor. In certain embodiments, the chimeric antigen receptor comprises a binding domain which binds to: CD19; CD20; CD22; ROR1; mesothelin; CD33/IL3Ra; c-Met; PSMA; Glycolipid F77; EGFRvIII; GD-2; NY-ESO-1; MAGE A3, or combinations thereof. In certain embodiments, the chimeric antigen receptor comprises a binding domain which binds to CD19.

### Definitions

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof

The terms “determining”, “measuring”, “evaluating”, “assessing,” “assaying,” and “analyzing” are often used interchangeably herein to refer to forms of measurement and include determining if an element is present or not (for example, detection). These terms can include quantitative, qualitative or quantitative and qualitative determinations. Assessing is alternatively relative or absolute. “Detecting the presence of” includes determining the amount of something present, as well as determining whether it is present or absent.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. The disease can be cancer. In some cases, the subject is not necessarily diagnosed or suspected of being at high risk for the disease.

The term “target cells” refers to a type of cell, cell population, or composition of cells which are the desired cells to be collected, isolated, or separated by the present invention. Target cells represent cells that various procedures described herein require or are designed to purify, collect, engineer etc. What the specific cells are will depend on the context in which the term is used. For example, if the objective of a procedure is to isolate a particular kind of stem cell, that cell would be the target cell of the procedure. The terms “target cells” and “desired cells” are interchangeable and have the same meaning regarding the present invention. Target cells can exist in a genus-species relationship. For example, if target cells comprised leukocytes, the target cells would include T cells. Target cells can also vary or be stratified through the collection process. For example, target cells in first step can consist of leukocytes and target cells in second step can consist of natural killer cells (NK cells).

Conversely, “non-target cells” comprise a type of cell, cell population, or composition of cells which are not the desired cells to be separated by the present invention. For example, if the target cells consist of T cells, in a leukocyte sample also comprising B cells and T cells, the B cells would be classified as a non-target cell. As another example, non-target cells can be cells that function in immunosuppression. the term “immunosuppression” refers to one or more cells, proteins, molecules, compounds or complexes providing inhibitory signals to assist in controlling or suppressing an immune response. For example, immunosuppression components include those molecules that partially or totally block immune stimulation; decrease, prevent or delay immune activation; or increase, activate, or up regulate immune suppression. “Controlling or suppressing an immune response,” as used herein, means reducing any one or more of antigen presentation, T cell activation, T cell proliferation, T cell effector function, cytokine secretion or production, and target cell lysis. Such modulation, control or suppression can promote or permit the persistence of a hyperproliferative disease or disorder (e.g., cancer, chronic infections).

The term “collect” generally refers to certain cell types and cell populations that have been enriched, separated, contained, isolated, etc. “Collected cells” refer to cells that have been subjected to enrichment, separation, containment, isolation, etc.

The term “leukocyte” is used interchangeably with the term “white blood cells” (“WBCs”). These terms include mononuclear agranulocytes, which include, e.g., monocytes, dendritic cell precursors, and lymphocytes, as well as polymorphonuclear granulocytes with segmented nuclei and cytoplasmic granules, including neutrophils, eosinophils, basophils, and mast cells.

The term “immune cell” refers generally to cells of the immune system. Immune cells are derived from myeloid or lymphoid cell linages.

The term “immune effector cell” refers to a cell that is involved in an immune response, e.g., in the promotion of an immune effector response. Examples of immune effector cells include T cells, e.g., alpha/beta T cells and gamma/delta T cells, B cells, natural killer (NK) cells, natural killer T (NKT) cells, mast cells, and myeloid-derived phagocytes.

The term “immune effector function or immune effector response,” as that term is used herein, refers to function or response, e.g., of an immune effector cell, that enhances or promotes an immune attack of a target cell. E.g., an immune effector function or response refers a property of a T or NK cell that promotes killing or the inhibition of growth or proliferation, of a target cell. In the case of a T cell, primary stimulation and co-stimulation are examples of immune effector function or response.

The term “effector function” refers to a specialized function of a cell. Effector function of a T cell, for example, may be cytolytic activity or helper activity including the secretion of cytokines.

The term “myeloid cell” refers to terminally differentiated cells of the myeloid lineage. These cells include neutrophils, eosinophils and monocytes/macrophages. In one embodiment of any aspect of the present invention, the myeloid cell is a neutrophil, eosinophil or monocyte/macrophage.

The term “macrophage” and/or “macrophage-like cells” generally refers to macrophages, monocytes, and cells of macrophage/monocyte lineage including but not limited to dendritic cells, and any other similar cells which perform the functions generally associated with macrophages, such as antigen presentation to other classes of immune cells such as T-cells and B-cells in order to sensitize these cells to a particular target, including but not limited to viruses, bacterial cells, other foreign cells, cancer cells, and other undesired proliferating cells.

The term “lymphocyte” The term includes natural killer (NK) cells, T cells, or B cells. NK cells are a type of cytotoxic (cell toxic) lymphocyte that represent a major component of the inherent immune system. NK cells reject tumors and cells infected by viruses. It works through the process of apoptosis or programmed cell death.

The term “natural killer (NK) cells” refers to cells of the immune system that kill target cells in the absence of a specific antigenic stimulus, and without restriction according to MHC class. Target cells may be tumor cells or cells harboring viruses. NK cells are characterized by the presence of CD56 and the absence of CD3 surface markers.

The term “endogenous cells” is used to refer to cells derived from a donor (or the patient), as distinguished from cells from a cell line. Endogenous cells are generally heterogeneous populations of cells from which a specific cell type can be isolated or enriched. Endogenous cells may be intended for autologous or allogeneic treatment of a patient.

The term “placental cells” refers to nucleated cells, e.g., total nucleated cells, isolated from, or isolatable from, placental perfusate. The term “placental perfusate” means perfusion solution that has been passed through at least part of a placenta, e.g., a human placenta, e.g., through the placental vasculature, including a plurality of cells collected by the perfusion solution during passage through the placenta.

The term “T cell” refers to a subset of lymphocytic cells that are present in PBMC and express a surface marker of “CD3” (T-cell receptor). Unless otherwise indicated T cells are intended to include CD4⁺ (i.e., T-helper cells) and CD8⁺ (i.e., cytotoxic killer cells).

The term “naïve T cell” is a T cell that has differentiated in bone marrow and successfully undergone the positive and negative processes of central selection in the thymus. A naïve T cell is considered mature but is distinguished from activated T cells or memory T cells, as it is thought not to have yet encountered cognate antigen in the periphery.

The terms “Treg” or “regulatory T cell” refer to CD4+ T cells that suppresses CD4+CD25− and CD8+ T cell proliferation and/or effector function, or that otherwise down-modulate an immune response. Notably, Treg may down-regulate immune responses mediated by Natural Killer cells, Natural Killer T cells as well as other immune cells. Tregs can also be Foxp3+.

The term “memory T cell” is a specific type of infection-fighting T-cell that can recognize foreign invaders such as bacteria or viruses that were previously encountered by the cell during a prior infection or vaccination. At a second encounter with the invader, memory T cells can reproduce to mount a faster and stronger immune response than the first time the immune system responded to the invader. Central memory T cells are those that are long-lived and seed future effector T cell populations and can be identified by one or more of CCR7+CD45RA−CD45RO+CD62L+CD27+.

The term “apheresis” refers to a procedure in which blood from a patient or donor is at least partially separated from some of its components. More specific terms are “plateletpheresis” (referring to the separation of platelets) and “leukapheresis” (referring to the separation of leukocytes). In this context, the term “separation” refers to the obtaining of a product that is enriched in a particular component compared to whole blood and does not mean that absolute purity has been attained.

The term “blood-related sample” refers to blood samples including whole-blood samples as well as samples derived from whole blood by the addition or removal of one or more cell types or chemical substances.

The term “adjusting the concentration of red blood cells” encompasses any method that changes the concentration of red blood cells in sample to achieve the stated concentration. Such methods include those that are deployed to specifically remove the red blood cells based on size, density or surface markers to achieve the stated concentration. Additional methods to adjust red blood cells are those that comprise dilution or buffer exchange into a lesser or greater amount of buffer from the starting buffer or concentration by a microfluidic device, centrifugation, or sedimentation.

The term “non-target cells” refer to any cell or cell type that is not red blood cells, platelets or the target cell type. For example, non-target cells may comprise regulatory T cells granulocytes, or any other cell-type that may have a negative effect on the target cell or cell types that while not deleterious to the target cells are not necessary for a down-stream therapeutic application.

The term “CAR” is an acronym for “chimeric antigen receptor.” Chimeric antigen receptors generally comprise a targeting domain that may, for example, be derived from the Fab region of an antibody (e.g., an scFv); a transmembrane domain; and one or more intracellular signaling domains CARs can be suitably expressed by a variety of cell types such as T cells (CAR T-cells), NK cells (CAR NK cells), or macrophages.

“CAR cell therapy” refers to any method in which a disease is treated with cells expressing a CAR. Diseases that may be treated include hematological and solid tumor cancers, autoimmune diseases and infectious diseases.

The term “carrier” refers an agent, e.g., a bead, or particle, made of either biological or synthetic material that is added to a preparation for the purpose of binding directly or indirectly (i.e., through one or more intermediate cells, particles or compounds) to some or all of the compounds or cells present. Carriers may be made from a variety of different materials, including DEAE-dextran, glass, polystyrene plastic, acrylamide, collagen, and alginate and will typically have a size of 1-1000 μm. They may be coated or uncoated and have surfaces that are modified to include affinity agents (e.g., antibodies, activators, haptens, aptamers, particles or other compounds) that recognize antigens or other molecules on the surface of cells. The carriers may also be magnetized, and this may provide an additional means of purification to complement DLD and they may comprise particles (e.g., Janus or Strawberry-like particles) that confer upon cells or cell complexes non-size related secondary properties. For example, the particles may result in chemical, electrochemical, or magnetic properties that can be used in downstream processes, such as magnetic separation, electroporation, gene transfer, and/or specific analytical chemistry processes. Particles may also cause metabolic changes in cells, activate cells or promote cell division.

Carriers may bind “in a way that promotes DLD separation.” This term, refers to carriers and methods of binding carriers that affect the way that, depending on context, a cell, protein or particle behaves during DLD. Specifically, “binding in a way that promotes DLD separation” means that: a) the binding must exhibit specificity for a particular target cell type, protein or particle; and b) must result in a complex that provides for an increase in size of the complex relative to the unbound cell, protein or particle. In the case of binding to a target cell, there must be an increase of at least 2 μm (and alternatively at least 20, 50, 100, 200, 500 or 1000% when expressed as a percentage). In cases where therapeutic or other uses require that target cells, proteins or other particles be released from complexes to fulfill their intended use, then the term “in a way that promotes DLD separation” also requires that the complexes permit such release, for example by chemical or enzymatic cleavage, chemical dissolution, digestion, due to competition with other binders, or by physical shearing (e.g., using a pipette to create shear stress) and the freed target cells, proteins or other particles must maintain activity; e.g., therapeutic cells after release from a complex must still maintain the biological activities that make them therapeutically useful.

The terms “Isolate” and “purify” unless otherwise indicated, are synonymous and refer to the enrichment of a desired product relative to unwanted material. The terms do not necessarily mean that the product is completely isolated or completely pure. For example, if a starting sample had a target cell that constituted 2% of the cells in a sample, and a procedure was performed that resulted in a composition in which the target cell was 60% of the cells present, the procedure would have succeeded in isolating or purifying the target cell.

The term “Deterministic Lateral Displacement” or “DLD” refers to a process in which particles are deflected on a path through an array, deterministically, based on their size in relation to some of the array parameters. This process can be used to separate cells, which is generally the context in which it is discussed herein. However, it is important to recognize that DLD can also be used to concentrate cells and for buffer exchange. Processes are generally described herein in terms of continuous flow (DC conditions; i.e., bulk fluid flow in only a single direction). However, DLD can also work under oscillatory flow (AC conditions; i.e., bulk fluid flow alternating between two directions).

The “critical size” or “predetermined size” of particles passing through an obstacle array describes the size limit of particles that are able to follow the laminar flow of fluid. Particles larger than the critical size can be ‘bumped’ from the flow path of the fluid while particles having sizes lower than the critical size (or predetermined size) will not necessarily be so displaced. When a profile of fluid flow through a gap is symmetrical about the plane that bisects the gap in the direction of bulk fluid flow, the critical size can be identical for both sides of the gap; however, when the profile is asymmetrical, the critical sizes of the two sides of the gap can differ.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Exemplary Embodiments

Among the exemplary embodiments disclosed herein are:

Provided are methods and compositions for use in processing a blood related sample comprising: (a) providing a blood related sample comprising one or more target cells, platelet cells, red blood cells, and a hematocrit of at least about 2%; and (b) reducing a number of the platelet cells in the blood related sample while maintaining a ratio of the red blood cells to the one or more target cells greater than about 50:1 to produce a reduced platelet blood related sample comprising the one or more target cells.

In some embodiments, the blood related sample comprises a hematocrit of greater than about 4%. In some embodiments, the blood related sample comprises a hematocrit of less than about 30%.

In some embodiments, the blood related sample is a leukapheresis product. In some embodiments, the reduced platelet blood related sample comprises a ratio of platelets to target cells of less than about 500:1. In certain embodiments, the reduced platelet blood related sample comprises a ratio of platelets to target cells of less than about 100:1.

In certain embodiments, the reduced platelet blood related sample comprises a ratio of platelets to target cells of less than about 10:1.

In some embodiments, the reduced platelet blood related sample comprises a ratio of platelets to target cells of less than about 5:1. In some embodiments, the red blood cells are maintained at a ratio of red blood cells to target cells of greater than about 100:1.

In certain embodiments, the red blood cells are maintained at a ratio of red blood cells to target cells of greater than about 250:1. In certain embodiments, the red blood cells are maintained at a ratio of red blood cells to target cells of greater than about 500:1. In certain embodiments, the red blood cells are maintained at a ratio of red blood cells to target cells of no greater than about 1,000:1.

In some embodiments, the method further comprises removing one or more non-target cells from the blood related sample and/or the reduced platelet blood related sample. In certain embodiments, the one or more non-target cells comprise immune suppressive cells. In certain embodiments, the immune suppressive cells are regulatory T cells. In certain embodiments, the immune suppressive cells are regulatory B cells. In certain embodiments, the immune suppressive cells comprise myeloid derived suppressor cells. In some embodiments, the target cell is a T cell and the non-target cells comprise leukocytes other than the T cell.

In some embodiments, the non-target cells are removed by an affinity-based method. In certain embodiments, the affinity-based method targets a molecule on the cell surface of the non-target cells. In some embodiments, the affinity-based method comprises the use of an antibody. In certain embodiments, the antibody is conjugated to biotin, streptavidin, a fluorescent moiety, or a magnetic material.

In some embodiments, the methods comprise adding an anticoagulant to the blood related sample. In some embodiments, the blood related sample is a human blood related sample. In some embodiments, the blood related sample is collected from an individual afflicted with a cancer or a tumor or an HLA matched individual to the individual afflicted with a cancer or a tumor. In certain embodiments, the blood related sample is collected from an individual afflicted with a cancer or a tumor. In some embodiments, the reducing the number of the platelet cells from the blood related sample comprises use of a method which uses an affinity reagent, a deterministic lateral displacement method, a method which uses a density media, an acoustophoretic method, or a dielectrophoretic method. In certain embodiments, the reducing the number of the platelet cells from the blood related sample uses a method comprising deterministic lateral flow.

In some embodiments, the method further comprises isolating the one or more target cells from the reduced platelet blood related sample to produce one or more isolated target cells. In some embodiments, the one or more target cells comprise peripheral blood mononuclear cells. In some embodiments, the one or more target cells comprise a stem cell, a lymphoid cell, or a myeloid cell. In certain embodiments, the stem cell is a hematopoietic stem cell. In certain embodiments, the lymphoid cell is a T cell. In certain embodiments, the T cell displays a naïve phenotype. In certain embodiments, the T cell displays a central memory phenotype. In certain embodiments, the lymphoid cell is a natural killer cell or a natural killer T cell. In certain embodiments, the myeloid cell is a dendritic cell. In certain embodiments, the myeloid cell is a macrophage cell. In some embodiments, the one or more target cells are isolated by a method which uses an affinity reagent, a deterministic lateral displacement method, a method which uses a density media, an acoustophoretic method, or a dielectrophoretic method. In some embodiments, the one or more target cells are isolated by a method which uses an affinity reagent. In some embodiments, the one or more target cells are isolated using deterministic lateral displacement.

In some embodiments, the method further comprises culturing the one or more target cells of the reduced platelet blood related sample or the one or more isolated target cells. In some embodiments, the method further comprises genetically engineering the one or more target cells of the reduced platelet blood related sample or the one or more isolated target cells. In certain embodiments, the genetic engineering comprises rendering the one or more target cells transgenic for a chimeric antigen receptor. In certain embodiments, the genetic engineering comprises rendering the one or more target cells transgenic for a recombinant T cell receptor. In some embodiments, the method further comprises comprising activating the one or more target cells prior to or after the genetic engineering.

Further provided are compositions, for example, provided are cell populations comprising one or more target cells, platelet cells and red blood cells, the target cells at a ratio of platelets to target cells less than about 500:1 and at a ratio of red blood cells to target cells of greater than about 50:1. In some embodiments, the target cells comprise human cells. In some embodiments, the target cells, platelet cells, and red blood cells comprise human cells.

In some embodiments, the ratio of platelets to target cells is less than about 100:1. In some embodiments, the ratio of platelets to target cells is less than about 10:1. In some embodiments, the ratio of platelets to target cells is less than about 5:1. In some embodiments, the ratio of red blood cells to target cells is greater than about 100:1. In some embodiments, the ratio of red blood cells to target cells is greater than about 250:1. In some embodiments, the ratio of red blood cells to target cells is greater than about 500:1. In some embodiments, the ratio of red blood cells to target cells is greater than about 1,000:1.

In some embodiments, the one or more target cells comprise peripheral blood mononuclear cells. In some embodiments, the one or more target cells comprise a stem cell, a lymphoid cell, or a myeloid cell. In some embodiments, the stem cell is a hematopoietic stem cell. In some embodiments, the lymphoid cell is a T cell. In some embodiments, the T cell displays a naïve phenotype. In some embodiments, the T cell displays a central memory phenotype. In some embodiments, the lymphoid cell is a natural killer cell or a natural killer T cell. In some embodiments, the myeloid cell is a dendritic cell. In some embodiments, the myeloid cell is a macrophage cell. In some embodiments, the one or more target cells comprise an exogenous nucleic acid encoding a chimeric antigen receptor or a recombinant T cell receptor. In some embodiments, the one or more target cells comprises an activated T cell. In some embodiments, the cell population is substantially free of one or more immune suppressive cells. In some embodiments, the immune suppressive cells are regulatory T cells. In some embodiments, the immune suppressive cells are regulatory B cells. In some embodiments, the immune suppressive cells comprise myeloid derived suppressor cells. In some embodiments, the one or more target cells possess the capacity to divide at least 3 time before exhaustion.

Disclosed are also processes for obtaining purified target cells from a blood related sample, wherein the blood related sample comprises target cells and red blood cells, the process comprising the steps of: (a) collecting the blood related sample from a patient; (b) removing platelets from the blood related sample collected in step (a); (c) optionally removing specific cells, other than platelets, from the sample prepared in step (b); (d) removing the red blood cells from the target cells after step b), or, if performed, after step (c) to obtain purified target cells; wherein, prior to step d, the red blood cell concentration in the blood related sample is maintained at, or adjusted to, at least 1×10⁴ red blood cells per microliter (μL).

In some embodiments, the patient is administered an anticoagulant for 1-10 days prior to the collection of the blood related sample.

In some embodiments, prior to step d), the red blood cell concentration in the blood related sample is maintained at, or adjusted to, a concentration of at least 1×10⁵ red blood cells per microliter (μL). In some embodiments, prior to step d), the red blood cell concentration in the blood related sample is maintained at, or adjusted to, a concentration of at least 5×10⁵ red blood cells per microliter (μL). In some embodiments, prior to step d), the red blood cell concentration in the blood related sample is maintained at, or adjusted to, a concentration of at least 1×10⁶ red blood cells per microliter (μL). In some embodiments, prior to step d), the red blood cell concentration in the blood related sample is maintained at, or adjusted to, a concentration of at least 5×10⁶ red blood cells per microliter (μL).

In some embodiments, the anticoagulant is added during the collection of blood in step a) using an in-line mixer. In some embodiments, the anticoagulant is a divalent metal chelator.

In some embodiments, the removal of platelets is initiated within 12 hours after the collection of blood is complete. In some embodiments, the removal of platelets is initiated within 6 hours after the collection of blood is complete. In some embodiments, the removal of platelets is initiated within 3 hours after the collection of blood is complete. In some embodiments, the removal of platelets is initiated within 1 hour after the collection of blood is complete. In some embodiments, the removal of platelets is initiated within 30 minutes after the collection of blood is complete. In some embodiments, the primary objective is the removal of platelets rather that maintaining a high yield of target cells. In some embodiments, in step b), platelets are removed by size, density, electric charge, acoustic properties, or any combination of these parameters on a microfluidic device or series of devices.

In some embodiments, in step b), platelets are removed by Deterministic Lateral Displacement (DLD) on a microfluidic device, wherein the device comprises:

at least one channel extending from a sample inlet to one or more fluid outlets, wherein the channel is bounded by a first wall and a second wall opposite from the first wall;

an array of obstacles arranged in rows in the channel, each subsequent row of obstacles being shifted laterally with respect to a previous row, and wherein the obstacles are disposed in a manner such that, when the blood related sample is applied to an inlet of the device and fluidically passed through the channel, target cells flow to one or more collection outlets where an enriched product is collected and platelets flow to one more waste outlets that are separate from the collection outlets.

In some embodiments, in step d), red blood cells are removed by size; density; electric charge; acoustic properties or any combination of these parameters on a microfluidic device.

In some embodiments, in step d), red blood cells are removed from target cells by Deterministic Lateral Displacement (DLD) on a microfluidic device, wherein the device comprises:

at least one channel extending from a sample inlet to one or more fluid outlets, wherein the channel is bounded by a first wall and a second wall opposite from the first wall;

an array of obstacles arranged in rows in the channel, each subsequent row of obstacles being shifted laterally with respect to a previous row, and wherein the obstacles are disposed in a manner such that, when a sample comprising red blood cells and target cells is applied to an inlet of the device and fluidically passed through the channel, target cells flow to one or more collection outlets where an enriched product is collected and red blood cells flow to one more waste outlets that are separate from the collection outlets.

In some embodiments, after the purified target cells are obtained in step d) they are genetically engineered to have a desired phenotype. In some embodiments, after purified target cells are obtained or genetically engineered, they are expanded in culture. In some embodiments, after purified target cells are obtained or genetically engineered, they are used to treat the same patient from which the blood sample was obtained. In some embodiments, the target cells are leukocytes, stem cells, immune or hematopoietic cells. In some embodiments, the target cells are T cells.

Disclosed are processes for producing CAR T cells, comprising: (a) collecting a blood related sample comprising T cells from a patient; (b) removing platelets from the blood related sample collected in step a) (c) removing contaminant cells, other than platelets, from the sample prepared in step b); (d) removing the red blood cells from the T cells after step c) to obtain purified T cells; (e) genetically engineering the purified T cells to express the chimeric antigen receptors (CARs) on their surface, wherein, prior to step d), the red blood cell concentration in the blood related sample is maintained at, or adjusted to, at least 1×10⁴ red blood cells per microliter (μL).

In some embodiments, either before or after the purified T cells are genetically engineered, they are expanded in cell culture. In some embodiments, the purified T cells are combined with a T cell activator one to 1-5 days before being genetically engineered, but no activator is added to the T cells prior to that time. In some embodiments, the cells are activated for a period of 1-5 days before being genetically engineered. In some embodiments, the T cell activator is added within 24 hours after purified T cells are obtained. In some embodiments, the cells are genetically engineered by viral transformation wherein a viral vector is added to purified T cells either sequentially or simultaneously with a T cell activator, cells are washed after virus integration and then the transformed cells are immediately reinfused into the patient.

In some embodiments, the cells are genetically engineered by viral transformation wherein activator, a viral vector and growth factors are added to purified T cells in one step and the cells are cultured ex-vivo, for subsequent re-infusion.

In some embodiments, after culturing, cells are reinfused into the patient without being frozen. In some embodiments, after culturing, cells are frozen before being reinfused into the patient. In some embodiments, the T cell activator is a cytokine or antibody the activator may be used either in solution or immobilized on a bead or carrier. In some embodiments, the T cell activator is a magnetic bead coated with anti-CD3/CD28 antibodies. In some embodiments, the T cell activator is a T cell specific antibody or nanobead carrying a T cell specific antibody. In some embodiments, the T cell activator is a nano-matrix or soluble reagent that activate.

In some embodiments, naive T cells are isolated by immunoselective separation, non-naive T cells are removed by immunoselective separation and the naive T cells are activated either before separation (together with other T cells) or individually after immuno separation. In some embodiments, the T cell activator is removed from the T cells prior to genetic engineering. In some embodiments, the T cell activator is not removed from the T cells prior to genetic engineering. In some embodiments, the purified T cells are concentrated before being genetically engineered. In some embodiments, cells are concentrated by DLD on a microfluidic device.

In some embodiments, the CARs comprise a) an extracellular region comprising antigen binding domain; b) a transmembrane region; c) an intracellular region and wherein the CAR T cells optionally comprise one or more recombinant sequences that provide the cells with a molecular switch that, when triggered, reduce CAR T cell number or activity. In some embodiments, the T cells are derived from a patient with cancer, an autoimmune disease or an infectious disease. In some embodiments, in step c), T regulatory cells are removed. In some embodiments, the T regulatory cells are removed using CD 25 as a marker. In some embodiments, the T regulatory cells are removed using microbeads with antibodies recognizing CD 25 on their surface. In some embodiments, in step c), activated T cells are removed. In some embodiments, the activated cells are removed using CD69 or CD 25 as a marker.

In some embodiments, in step c), antigen presenting cells are removed.

In some embodiments, the antigen presenting cells are B cells. In some embodiments, the B cells are removed using CD19, CD10 or CD20 as a marker. In some embodiments, the B cells are removed using microbeads with antibodies recognizing CD19, CD10 or CD20 on their surface.

In some embodiments, in step c), dendritic cells are removed. In some embodiments, the dendritic cells are removed using CLEC9a, CD1c, CD11c, or CD141, CD14, CD205, CD83, BDCA1, or BDCA2 as a marker.

In some embodiments, in step c), granulocytes are removed. In some embodiments, the granulocytes are removed using CD16 and optionally CD66, and/or CD11b, as a marker.

In some embodiments, the patient is administered an anticoagulant for 1-10 days prior to the collection of the blood related sample.

In some embodiments, prior to step d), the red blood cell concentration in the blood related sample is maintained at, or adjusted to, a concentration of at least 1×10⁵ red blood cells per microliter (microliter (μL)). In some embodiments, prior to step d), the red blood cell concentration in the blood related sample is maintained at, or adjusted to, a concentration of at least 5×10⁵ red blood cells per microliter (microliter (μL)). In some embodiments, prior to step d), the red blood cell concentration in the blood related sample is maintained at, or adjusted to, a concentration of at least 1×10⁶ red blood cells per microliter (microliter (μL)). In some embodiments, prior to step d), the red blood cell concentration in the blood related sample is maintained at, or adjusted to, a concentration of at least 5×10⁶ red blood cells per microliter (microliter (μL)).

In some embodiments, anticoagulant is added during the collection of blood in step a) using an in-line mixer. In some embodiments, the anticoagulant is a divalent metal chelator.

In some embodiments, the removal of platelets is initiated within 12 hours after the collection of blood is complete. In some embodiments, he removal of platelets is initiated within 6 hours after the collection of blood is complete. In some embodiments, the removal of platelets is initiated within 3 hours after the collection of blood is complete. In some embodiments, the removal of platelets is initiated within 1 hour after the collection of blood is complete. In some embodiments, the removal of platelets is initiated within 30 minutes after the collection of blood is complete. In some embodiments, T cell activator is added within 24 hours after the collection of blood is complete. In some embodiments, in step b), platelets are removed by size, density, electric charge, acoustic properties, or any combination of these parameters on a microfluidic device or series of devices.

In some embodiments, in step b), platelets are removed by Deterministic Lateral Displacement (DLD) on a microfluidic device, wherein the device comprises: at least one channel extending from a sample inlet to one or more fluid outlets, wherein the channel is bounded by a first wall and a second wall opposite from the first wall; an array of obstacles arranged in rows in the channel, each subsequent row of obstacles being shifted laterally with respect to a previous row, and wherein the obstacles are disposed in a manner such that, when a sample comprising red blood cells and T cells is applied to an inlet of the device and fluidically passed through the channel T cells flow to one or more collection outlets where an enriched product is collected and platelets flow to one more waste outlets that are separate from the collection outlets.

In some embodiments, in step d), red blood cells are platelets are removed by size, density, electric charge, acoustic properties, or any combination of these parameters on a microfluidic device or series of devices.

In some embodiments, in step d), red blood cells are removed from target cells by Deterministic Lateral Displacement (DLD) on a microfluidic device, wherein the device comprises: at least one channel extending from a sample inlet to one or more fluid outlets, wherein the channel is bounded by a first wall and a second wall opposite from the first wall; an array of obstacles arranged in rows in the channel, each subsequent row of obstacles being shifted laterally with respect to a previous row, and wherein the obstacles are disposed in a manner such that, when a sample comprising red blood cells and T cells is applied to an inlet of the device and fluidically passed through the channel, T cells flow to one or more collection outlets where an enriched product is collected and red blood cells flow to one more waste outlets that are separate from the collection outlets.

In some embodiments, after T cells are genetically engineered in step e), T cells are separated from transformation agents and transferred into stabilization buffer, growth medium or cell culture medium.

In some embodiments, in step d), red blood cells are removed from target cells by Deterministic Lateral Displacement (DLD) on a microfluidic device, wherein the device comprises:

at least one channel extending from a sample inlet to one or more fluid outlets, wherein the channel is bounded by a first wall and a second wall opposite from the first wall;

an array of obstacles arranged in rows in the channel, each subsequent row of obstacles being shifted laterally with respect to a previous row, and wherein the obstacles are disposed in a manner such that, when a sample comprising transformation agents and T cells is applied to an inlet of the device and fluidically passed through the channel, T cells flow to one or more collection outlets where an enriched product is collected and transformation agents flow to one more waste outlets that are separate from the collection outlets.

In some embodiments, centrifugation is not performed during the process. In some embodiments, cells are not frozen at any point in the process.

Further disclosed are methods for obtaining target cells from a blood related sample, wherein the blood related sample comprises target cells, platelet cells, and red blood cells, the process comprising the steps of: (a) reducing platelets from the blood related sample, thereby providing a reduced platelet blood related sample; and (b) reducing or adjusting red blood cells of the reduced platelet blood related sample, thereby providing an adjusted red blood cell, reduced platelet blood related sample; wherein the adjusted red blood cell, reduced platelet blood related sample comprises at least about 1×10³ red blood cells per microliter to about 1×10⁷per microliter.

In some embodiments, the methods comprise removing one or more non-target cells from the reduced platelet blood related sample or the adjusted red blood cell, reduced platelet blood related sample. In some embodiments, the non-target cells are selected from the list consisting of regulatory T cells, regulatory B cells, and granulocytes. In some embodiments, the non-target cells are regulatory T cells. In some embodiments, the non-target cells are regulatory B cells. In some embodiments, the non-target cells are granulocytes.

Provided are methods for isolation and expanding target cells from a sample comprising (a) generating a composition comprising target cells and red blood cells, and (b) expanding the target cells to generate an expanded target cell population. In some embodiments, the method comprises, prior to (a), removing platelets from the sample to generate a target cell composition that further comprises reduced platelet number. In some embodiments, the sample comprises non-target cells wherein the non-target cells are removed prior to (a). In some embodiments, non-target cells are removed from the target cell composition subsequent to (a). In some embodiments, the method further comprises, harvesting or isolating the expanded target cells. In some embodiments, expanded T cells are harvested 4, 5, 6, 7, or 8 days after expansion.

Further provided herein are methods for isolation and expanding T cells from a sample comprising (1) generating a composition comprising T cells and red blood cells (RBCs); and (2) expanding the T cells to generate an expanded T cell population. In some embodiments, the methods comprise generating a target cell composition comprising T cells and red blood cells, as disclosed herein, and expanding the T cells to generate an expanded T cell population. In some embodiments, the T cells are CD4+ or naïve CD4+ T cells. In some embodiments, the expanded T cell population comprises CD8+ T cells or CD8+ T memory cells. In some embodiments, non-target cells are removed prior to generating the target cell composition. In some embodiments, the non-target cells are removed after generating the target cell composition. In some embodiments, the target cell composition consists of T cells and red blood cells. In some embodiments, the method further comprises removing platelets prior to or as part of generating said target cell composition. In some embodiments, the method further comprises modifying the genome of the expanded T cell population to generate a T cell comprising an engineered T-cell receptor. In some embodiments, the engineered T-cell receptor comprises a chimeric antigen receptor. In some embodiments, the T cell comprising the engineered T-cell receptor in a chimeric antigen receptor T cell (CAR-T) or engineered T cell receptor T cell (TCR-T). In some embodiments, expanded T cells are harvested 4, 5, 6, 7, or 8 days after expansion.

Accordingly, provided herein are target cell compositions for use in generating engineered target cells, wherein the target cell compositions comprise red blood cells. In some embodiments, platelets are removed from the target cell composition. In some embodiments, platelets the target cell composition is also a reduced platelet target cell composition, wherein the number of platelets has been reduced, as disclosed herein. In some embodiments, the target cells are T cells. In certain embodiments, the T cells are CD4 +or CD8+. In some embodiments, the target cell compositions are for use in an expansion method (e.g. generating an expanded target cell composition or product), wherein the target cell compositions have not yet been subjected to an expansion reaction. In some embodiments, the expanded target cells comprise CD8+ T memory cells. In some embodiments, the CD8+ T cells are converted from CD4+ T cells or naïve CD4+ cells. In some embodiments, the target cell is a CD4+ T cells and the expanded target cell composition is CD8+ T memory cells.

The methods descried herein provide a number of functional benefits including a higher percentage of CD8+ T cells. The methods descried herein provide a number of functional benefits including a higher percentage of memory CD8+ T cells, including a percentage at least about 60%, 70%, 80% or greater. The methods descried herein provide a number of functional benefits including a higher absolute number of T cells, including a number of T cells that is at least about 2-fold, 3-fold, 4-fold, 5-fold, or 6-fold in excess of the numbers obtained using methods that do not maintain a critical concertation of RBCs during processing and/or expansion.

### EXAMPLES

The following examples are included for illustrative purposes only and are not intended to limit the scope of the invention.

### Example 1: Ordered Processing Of Blood Samples and Target Cell Compositions Comprising Red Blood Cells

Canonical processing of T cells (e.g. target cells) for therapeutic use comprises the isolation of white blood cells (WBC) from both plasma and red blood cells. As exemplified in FIG. 1A, the removal of red blood cells not only results in reduced protection from the shear stress forces, but also results in the increased cell to cell interactions that drive reduced viability and expansion capacities (e.g. less naïve CD4 cells are available for CAR engineering) of the T cells (e.g. target cells). Furthermore, the presence of platelets in a sample subject the T cells (e.g. target cells) to factors that drive reduced viability and expansion capacities (e.g. immunosuppressive factors). FIG. 1B exemplifies the solution and benefits to target cell processing methods and target cell compositions that comprise red blood cells. The presence of red blood cells insulates or cushions target cells from cell to cell interactions that drive reduced viability and expansion capacities (e.g. less naïve CD4 cells are available for CAR engineering) of the T cells (e.g. target cells). Such principles are exemplified and disclosed herein.

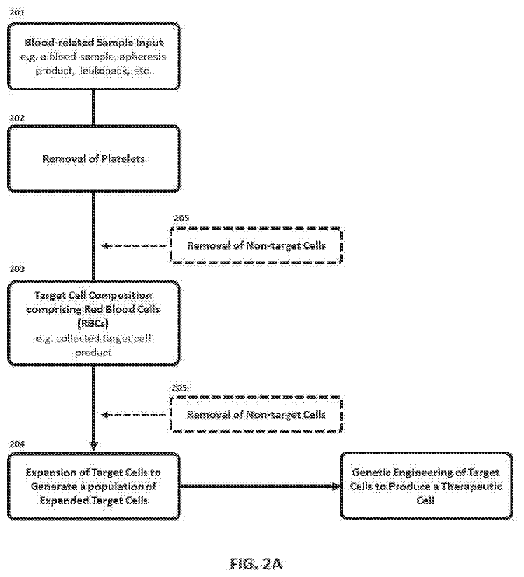

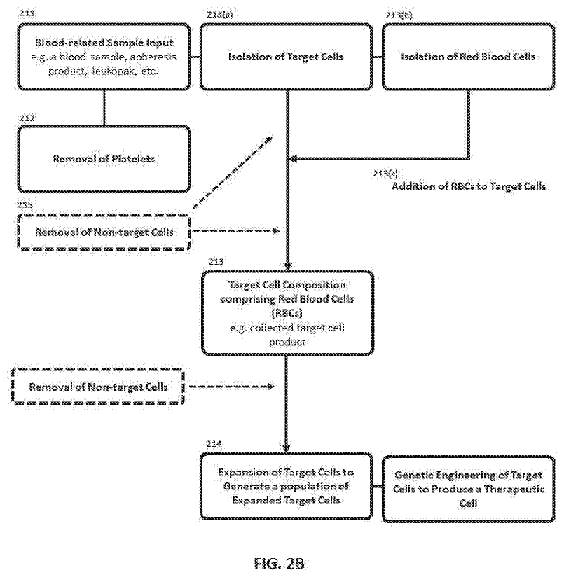

The generation of target cell compositions comprising target cells and red blood cells (RBC) can be achieved through an array of ordered processes wherein the resulting target cell composition (e.g. collected target cell product) comprises red blood cells. FIG. 2A illustrates exemplary steps wherein platelets are removed 202 from a sample (e.g. a blood-related sample) to produce a target cell composition comprising target cells (e.g. T cells) and red blood cells 203. Non-target cells (e.g. leukocytes that are not T cells or immunosuppressive cells) can be removed 205 (e.g. by positive or negative selection) at various steps. Target cells can then be expanded 204 to produce an expanded target cell population. Target expanded target cells can further be harvested 1-9 days post expansion, more preferably 4-8 days post expansion. Operations 201, 202, and 203 can take place across a single system (e.g. DLD) or multiple systems (e.g. density separation and DLD). Operations 201, 202, and 203 can also be performed more than once to achieve the desired collected cell product.

FIG. 2B illustrates exemplary steps wherein platelets, red blood cells, and target cells are collected 212, 213(a), and 213(b) from a sample (e.g. a blood-related sample). Red blood cells are then added 213(c) to the target cells to produce a target cell composition comprising target cells (e.g. T cells) and red blood cells 213. Non-target cells (e.g. leukocytes that are not T cells or that are immunosuppressive cells) can be removed 215 (e.g. by positive or negative selection) at various steps. Target cells can then be expanded 214 to produce an expanded target cell population. Target expanded target cells can further be harvest 1-9 days post expansion, more preferably 4-8 days post expansion. Operations 211, 212, and 213(a)-(c) can take place across a single system (e.g. DLD) or multiple systems (e.g. density separation and DLD). Operations 211, 212, and 213(a)-(c) can also be performed more than once to achieve the desired collected cell product.

### Example 2: Target Cell Compositions Comprising Red Blood Cells Possess Greater Expansion Capacities

A potential mechanism that supports the Order of Operations methods and compositions described is that upon apheresis and the removal of a substantial amount of RBC (going down from an hematocrit of about 35 to 3.0 or below), WBC have a higher probability of interaction with other blood cells, plasma components and platelets leading to their improper activation and WBC dysfunction. Such interaction occurring during and after the Apheresis. In other words, RBC may “buffer” or “protect” WBC from detrimental cell-cell interactions and soluble factors present in the Apheresis product.

Described in this example are experiments to test whether the presence of red blood cells (RBC), present at different concentrations (hematocrit) affect and/protect the basal activity of white blood cells (WBC) as measured by cell surface markers in WBC and their ability to perform a robust proliferation and expansion upon activation.

In order to evaluate the questions above Apheresis product (LRS) along with citrated whole blood from the same human donor were obtained, and processed to create samples of differing hematocrit with and without platelets.

In order to obtain purified RBC to spike into samples with platelets (Plasma) or without platelets (PlasmaLyte), WBC were removed from whole blood, first by centrifugation, followed by DLD of the RBC and then by a second centrifugation to substantially reduce remaining PLT and WBC in the RBC fraction. The resulting purified RBC were used to prepare different hematocrit solutions of 2.5%, 5.0%, 10%, and 20%.

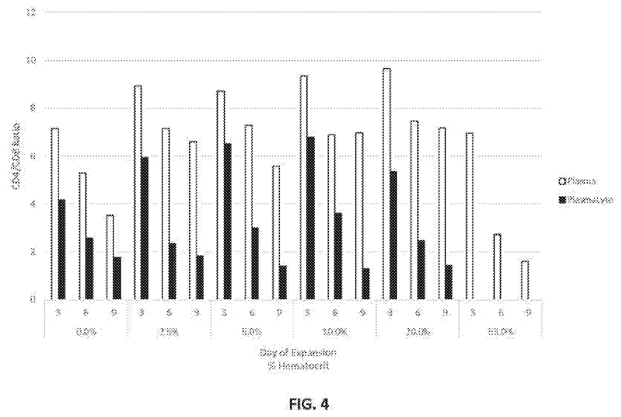

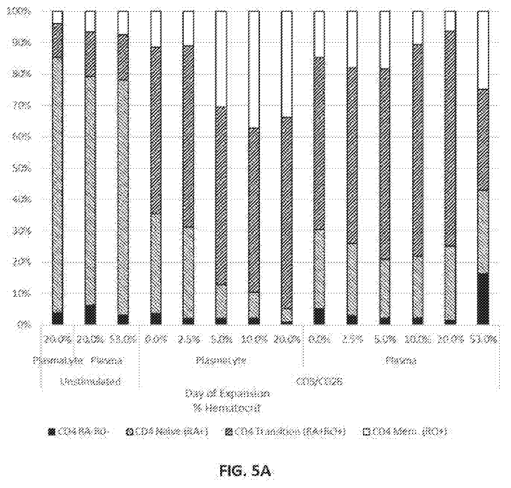

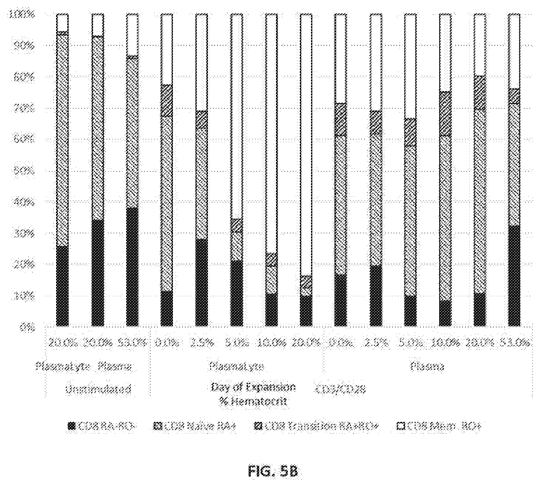

To generate samples with Plasma and platelets, a fraction of the LRS was processed to obtain the buffy coat by diluting the LRS 1:1 with PlasmaLyte A (PLA), and centrifugation. PLA is a sterile, pH buffered solution isotonic with blood plasma, which contains no blood cells such as RBC, platelets or WBC. First, the PLT-rich plasma and the buffy coat were removed (interface between the plasma and RBC) and passed through a DLD process. The RBC-free WBC in the product were reconstituted with the PLT-rich plasma (Samples denoted as plasma in FIGS. 4, 5A, and 5B).

To generate samples that were plasma and platelet free the LRS was softly spun to remove plasma and PLT, then the buffy coat was run on a DLD, thus removing both RBC and PLT from the WBC fraction, followed by resuspension in PLA. The resulting WBC do not have platelet or plasma components (Samples denoted as PlasmaLyte in in FIGS. 4, 5A, and 5B).

A fraction of LRS was kept in its native state, as a control (53% hematocrit).

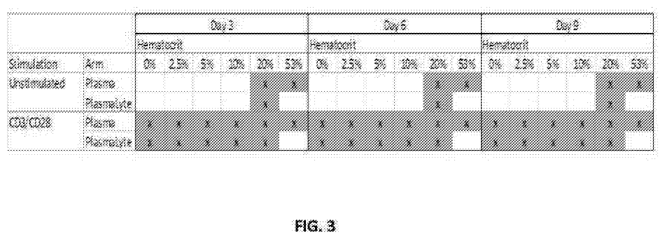

An experimental overview is shown in FIG. 3.

Ten million WBC, from the samples with plasma and platelets; and 5 million WBC from samples without plasma and platelets were dispensed in tubes containing either 0, 2.5, 5.0, 10 or 20% hematocrit (RBC from ARM A) and incubated for one hour at room temperature. After the incubation all tubes, including the control, were stimulated with T-cell activating magnetic beads (CD3/CD28) and incubated for one hour at 37° C. Upon completion the beads:cells complexes were removed and plated on cell culture wells at 0.5×10\hat{ }6 cells/ml of TexMac media containing 5.0 ng of IL-7 and IL-15 to induce the expansion of the activated cells.

Upon incubation with the different hematocrits cell aliquots were removed and stained with the indicated activation cocktails to assess their activation status and phenotype.

As shown by the data in FIG. 4, the PlasmaLyte samples (without platelets) showed a decrease in CD4/CD8 ratio indicating greater conversion of CD4+ to CD8+ T cells, a desirable phenotypic trait for many therapeutic T cell applications. Additionally, as shown in FIG. 5B, at day 6 post expansion with CD3 and CD28, CD8 cells showed higher conversion to a central memory phenotype (compare PlasmaLyte to Plasma) and increasing hematocrit had a positive effect on this conversion to central memory phenotype.

The data support that a method to recover the highest number of naïve and intact cells for T cell therapy will consist of a first step removing plasma components and platelets, then an optional step to remove unwanted blood cells like B, monocytes and granulocytes followed by a final step to remove RBC. This “Order of Operations” ensures recovery of the most intact and naïve WBC suitable for cell therapies and by consequence this method will yield the highest number of Tcm cells suitable for therapies.

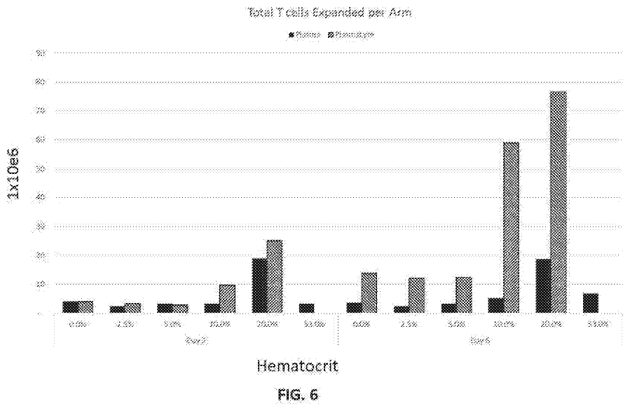

FIG. 6 shows the comparative increase in absolute numbers of T cells following costimualtion within each of the arms, and shows the superior expansion profile as a result of the combined effects of soluble factor removal and the intentional reduction in the frequency of T:Leucocyte interaction prior to intentional co-stimulation.

Analysis Cocktails: Day 0, 3, 6, 9: CD3/CD4/CD8/CD45RA/CD45RO/Live & Dead

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for processing a blood related sample comprising: (a) providing a blood related sample comprising one or more target cells, platelet cells, red blood cells; and (b) reducing a number of the platelet cells in the blood related sample while maintaining a ratio of the red blood cells to the one or more target cells greater than about 50:1 to produce a reduced platelet blood related sample comprising the one or more target cells.

2. The method of claim 1, wherein the blood related sample comprises a hematocrit of greater than about 2%.

3. The method of claim 1, wherein the blood related sample comprises a hematocrit of greater than about 4%.

4. The method of claim 1, wherein the blood related sample comprises a hematocrit of less than about 30%.

5. The method of claim 1, wherein the blood related sample is a leukapheresis product.

6. The method of any one of claims 1 to 5, wherein the reduced platelet blood related sample comprises a ratio of platelets to target cells of less than about 500:1.

7. The method of claim 6, wherein the reduced platelet blood related sample comprises a ratio of platelets to target cells of less than about 100:1.

8. The method of claim 6, wherein the reduced platelet blood related sample comprises a ratio of platelets to target cells of less than about 10:1.

9. The method of claim 6, wherein the reduced platelet blood related sample comprises a ratio of platelets to target cells of less than about 5:1.

10. The method of any one of claims 1 to 9, wherein the red blood cells are maintained at a ratio of red blood cells to target cells of greater than about 100:1.

11. The method of claim 10, wherein the red blood cells are maintained at a ratio of red blood cells to target cells of greater than about 250:1.

12. The method of claim 10, wherein the red blood cells are maintained at a ratio of red blood cells to target cells of greater than about 500:1.

13. The method of claim 10, wherein the red blood cells are maintained at a ratio of red blood cells to target cells of no greater than about 1,000:1.

14. The method of any one of claims 1 to 13, further comprising removing one or more non-target cells from the blood related sample and/or the reduced platelet blood related sample.

15. The method of claim 14, wherein the one or more non-target cells comprise immune suppressive cells.

16. The method of claim 15, wherein the immune suppressive cells are regulatory T cells.

17. The method of claim 15, wherein the immune suppressive cells are regulatory B cells.

18. The method of claim 15, wherein the immune suppressive cells comprise myeloid derived suppressor cells.

19. The method of any one of any one of claims 14 to 18, wherein the non-target cells are removed by an affinity-based method.

20. The method of claim 19, wherein the affinity-based method targets a molecule on the cell surface of the non-target cells.

21. The method of claim 19 or 20, wherein the affinity-based method comprises the use of an antibody.

22. The method of claim 21, wherein the antibody is conjugated to biotin, streptavidin, a fluorescent moiety, or a magnetic material.

23. The method of any one of claims 1 to 22, comprising adding an anticoagulant to the blood related sample.

24. The method of any one of claims 1 to 23, wherein the blood related sample is a human blood related sample.

25. The method of any one of claims 1 to 24, wherein the blood related sample is collected from an individual afflicted with a cancer or a tumor or an HLA matched individual to the individual afflicted with a cancer or a tumor.

26. The method of claim 25, wherein the blood related sample is collected from an individual afflicted with a cancer or a tumor.

27. The method of any one of claims 1 to 26, wherein reducing the number of the platelet cells from the blood related sample comprises use of a method which uses an affinity reagent, a deterministic lateral displacement method, a method which uses a density media, an acoustophoretic method, or a dielectrophoretic method.

28. The method of claim 27, wherein reducing the number of the platelet cells from the blood related sample uses a method comprising deterministic lateral flow.

29. The method of any one of claims 1 to 28, further comprising isolating the one or more target cells from the reduced platelet blood related sample to produce one or more isolated target cells.

30. The method of any one of claims 1 to 29, wherein the one or more target cells comprise peripheral blood mononuclear cells.

31. The method of any one of claims 1 to 29, wherein the one or more target cells comprise a stem cell, a lymphoid cell, or a myeloid cell.

32. The method of claim 30, wherein the stem cell is a hematopoietic stem cell.

33. The method of claim 30, wherein the lymphoid cell is a T cell.

34. The method of claim 33, wherein the T cell displays a naïve phenotype.

35. The method of claim 33, wherein the T cell displays a central memory phenotype.

36. The method of claim 30, wherein the lymphoid cell is a natural killer cell or a natural killer T cell.

37. The method of claim 30, wherein the myeloid cell is a dendritic cell.

38. The method of claim 30, wherein the myeloid cell is a macrophage cell.

39. The method of any one of claims 29 to 38, wherein the one or more target cells are isolated by a method which uses an affinity reagent, a deterministic lateral displacement method, a method which uses a density media, an acoustophoretic method, or a dielectrophoretic method.

40. The method of any one of claims 29 to 38, wherein the one or more target cells are isolated by a method which uses an affinity reagent.

41. The method of any one of claims 29 to 38, wherein the one or more target cells are isolated using deterministic lateral displacement.

42. The method of any one of claims, 1 to 41, further comprising culturing the one or more target cells of the reduced platelet blood related sample or the one or more isolated target cells.

43. The method of any one of claims, 1 to 41, further comprising genetically engineering the one or more target cells of the reduced platelet blood related sample or the one or more isolated target cells.

44. The method of claim 43, wherein the genetic engineering comprises rendering the one or more target cells transgenic for a chimeric antigen receptor.

45. The method of claim 43, wherein the genetic engineering comprises rendering the one or more target cells transgenic for a recombinant T cell receptor.

46. The method of any one of claims 43 to 45, further comprising activating the one or more target cells prior to or after the genetic engineering.

47. A cell population comprising one or more target cells, platelet cells and red blood cells, the target cells at a ratio of platelets to target cells less than about 500:1 and at a ratio of red blood cells to target cells of greater than about 50:1.

48. The cell population of claim 47, wherein the target cells comprise human cells.

49. The cell population of claim 47, wherein the target cells, platelet cells, and red blood cells comprise human cells.

50. The cell population of any one of claims 47 to 50, wherein the ratio of platelets to target cells is less than about 100:1.

51. The cell population of any one of claims 47 to 50, wherein the ratio of platelets to target cells is less than about 10:1.

52. The cell population of any one of claims 47 to 50, wherein the ratio of platelets to target cells is less than about 5:1.

53. The cell population of any one of claims 47 to 52, wherein the ratio of red blood cells to target cells is greater than about 100:1.

54. The cell population of any one of claims 47 to 52, wherein the ratio of red blood cells to target cells is greater than about 250:1.

55. The cell population of any one of claims 47 to 52, wherein the ratio of red blood cells to target cells is greater than about 500:1.

56. The cell population of any one of claims 47 to 52, wherein the ratio of red blood cells to target cells is greater than about 1,000:1.

57. The cell population of any one of claims 47 to 56, wherein the one or more target cells comprise peripheral blood mononuclear cells.

58. The cell population of any one of claims 47 to 56, wherein the one or more target cells comprise a stem cell, a lymphoid cell, or a myeloid cell.

59. The cell population of claim 58, wherein the stem cell is a hematopoietic stem cell.

60. The cell population of claim 58, wherein the lymphoid cell is a T cell.

61. The cell population of claim 60, wherein the T cell displays a naïve phenotype.

62. The cell population of claim 60, wherein the T cell displays a central memory phenotype.

63. The cell population of claim 58, wherein the lymphoid cell is a natural killer cell or a natural killer T cell.

64. The cell population of claim 58, wherein the myeloid cell is a dendritic cell.

65. The cell population of claim 58, wherein the myeloid cell is a macrophage cell.

66. The cell population of any one of claims 47 to 65, wherein the one or more target cells comprise an exogenous nucleic acid encoding a chimeric antigen receptor or a recombinant T cell receptor.

67. The cell population of claim 47, wherein the one or more target cells comprises an activated T cell.

68. The cell population of any one of claims 47 to 67, wherein the cell population is substantially free of one or more immune suppressive cells.

69. The cell population of claim 68, wherein the immune suppressive cells are regulatory T cells.

70. The cell population of claim 68, wherein the immune suppressive cells are regulatory B cells.

71. The cell population of claim 68, wherein the immune suppressive cells comprise myeloid derived suppressor cells.

72. The cell population of any one of claims 47 to 71, wherein the one or more target cells possess the capacity to divide at least 3 time before exhaustion.
